SEQUENCE LISTING (5 sequences)

SEQ ID NO: 1 - protein, 5 aa, Artificial Sequence
  Synthetic polypeptide
XPXXX

SEQ ID NO: 2 - protein, 15 aa, Artificial Sequence
  Synthetic polypeptide
VPGAGVPGXGVPGAG

SEQ ID NO: 3 - protein, 5 aa, Artificial Sequence
  Synthetic polypeptide
IPAVG

SEQ ID NO: 4 - protein, 5 aa, Artificial Sequence
  Synthetic polypeptide
VPGEG

SEQ ID NO: 5 - protein, 6 aa, Artificial Sequence
  Synthetic polypeptide
GFXGER

(19) United States
(12) Patent Application Publication
(10) Pub. No.: US 2014/0193477 A1
(43) Pub. Date: Jul. 10, 2014

(54) COLLAGEN BASED MATERIALS AND USES RELATED THERETO

(71) Applicants: Emory University, Atlanta, GA (US); Beth Israel Deaconess Medical Center, Inc., Boston, MA (US)

(72) Inventors: Elliot Chaikof, Newton, MA (US); Jeffrey Caves, Palo Alto, CA (US); Vivek Ashok Kumar, Houston, TX (US); Adam W. Martinez, Decatur, GA (US)

(73) Assignees: Emory University, Atlanta, GA (US); Beth Israel Deaconess Medical Center, Inc., Boston, MA (US)

(21) Appl. No.: 14/052,581

(22) Filed: Oct. 11, 2013

Related U.S. Application Data
(60) Provisional application No. 61/712,350, filed on Oct. 11, 2012.

(51) Int. Cl. A61L 27/44; A61L 27/54; A61L 27/24

(52) U.S. Cl. CPC A61L 27/44; A61L 27/24; A61L 27/54
USPC 424/443; 530/356; 514/773; 424/93.1; 424/549; 424/602; 428/221; 428/220

(57) ABSTRACT

This disclosure relates to materials fabricated from collagen and uses relates thereto. Typically, layers of collagen are stretched during a curing period and optionally coated or impregnated with an elastin like protein. In certain embodiments, these materials can be used in tissue repair or arranged into cylinders and utilized as a prosthetic vascular graft.

### RELATED APPLICATIONS

This application claims priority under 35 U.S.C. §119(e) to U.S. provisional application, U.S. Ser. No. 61/712,350, filed Oct. 11, 2012, which is incorporated herein by reference herein.

### GOVERNMENT SUPPORT

This invention was made with government support under grant R01 HL083867-05 awarded by the National Institutes of Health. The government has certain rights in the invention.

### BACKGROUND

After decades of investigation, small to medium (less than 4-7 mm) diameter prosthetic vascular grafts continue to occlude due to peri-anastomotic intimal hyperplasia, surface thrombogenicity, and failure to develop an endothelialized lumen. Intimal hyperplasia, the formation of pannus tissue with luminal narrowing, is driven in part by endothelial injury and mechanical mismatch between stiff prosthetics and a compliant native artery. Disrupted flow and shear stresses are also recognized factors. Vascular graft thrombogenicity results from protein and cell adsorption, thrombin and fibrin formation, and platelet activation and aggregation. Thus, there is a need to identify materials to construct vascular grafts that address these issues.

Materials indicated for vascular tissue engineering include animal-derived biopolymer gels such as collagen, fibrin, composites, and decellularized natural tissues. These materials are often integrated with appropriate stem cells or progenitor cells to recreate artificial tissues that mimic the natural environment. Many strategies remain hampered by prolonged in vitro culture times required by cells for the secretion of an organized, mechanically sound extracellular matrix (ECM). Thus, there is a need to identify improved materials.

Recombinant proteins derived from elastin sequences have been investigated. In particular, elastin-like protein triblock copolymers contain less endotoxin than clinical grade alginate. These protein triblocks can be molded or laminated due to a defined inverse transition temperature, above which the hydrophobic endblocks of the copolymer aggregate to produce a physically crosslinked hydrogel. Mechanical properties are tunable through adjustment of copolymer block size or sequence, or through implementation of processing conditions that alter the degree of microphase block mixing. Primate ex vivo shunt studies have confirmed that elastin-like protein polymers can serve as thromboresistant luminal coatings for small diameter ePTFE vascular grafts. See Jordan et al., Biomaterials, 2007, 28: 1191-1197.

Early vascular tissue engineering with collagen gels validated the concept of ECM protein scaffolds but lacked strength largely due to microstructural deviations from native collagen fibril orientation, architecture, and packing density. Several methods have been reported to create collagen matrices. Examples include methods using electrical gradients, Cheng et al., Biomaterials, 2008, 29:3278-3288 magnetic fields, Girton et al., Methods Mol Med, 1999, 18: 67-73; Torbet & Ronziere, Biochem J, 1984, 219, 1057-1059, microfluidics, Guo & Kaufman, Biomaterials, 2007, 28: 1105-1114, Lee et al., Biomed Microdevices, 2006, 8:35-41, Lanfer et al., Biomaterials, 2008, 29: 3888-3895, and patterned substrates, Zorlutuna et al., Biomacromolecules, 2009, 10:814-821. Cheng et al used an electric field to align collagen molecules. However, their technique destroys the native collagen structure and denatures the molecule, as demonstrated by the lack of D-periodicity. Lee et al. and Lanfer et al. have employed the use of microfluidics to align collagen. Lee et al., Biomed Microdevices, 2006, 8: 35-41, Lanfer et al., Biomaterials, 2008, 29:3888-3895, Lanfer et al., Biomaterials, 2009, 30:5950-5958, and Lanfer et al., Tissue Eng Part A, 2010, 16:1103-1113. These constructs have the potential to form density gradients across the sample due to viscous flow shear, and along the sample due to fibril polymerization prior to fully traversing the length of the channel. This results in inhomogeneity within samples. Vader et al., PLoS One, 2009, 4:e5902, describe strain-induced alignment in collagen gels.

Caves et al., Biomaterials, 2010, 31 (27), 7175-7182, disclose the use of microfiber composites of elastin-like protein matrix reinforced with synthetic collagen in the design of vascular grafts. See also Caves et al., Biomaterials, 2011, 32 (23), 5371-5379. References cited herein are not an admission of prior art.

### SUMMARY OF THE INVENTION

This disclosure relates to synthetic materials fabricated from collagen. In certain embodiments, the disclosure relates to materials comprising a stretched collagen matrix with D-periodicity. In certain embodiments, the collagen matrix is coated or impregnated with an elastin-like protein polymer through direct contact with or without crosslinking agents. In certain embodiments, these materials can be used in tissue repair (e.g., hernia repair) or arranged into cylinders and used as a prosthetic vascular grafts.

In some embodiments, a prosthetic vascular graft may have a diameter of about 0.5 mm to about 5 mm. For example, the diameter of a vascular graft may be about 0.5 mm, 1 mm, 1.5 mm, 2 mm, 2.5 mm, 3 mm, 3.5 mm, 4 mm, 4.5 mm, or 5 mm,

In certain embodiments, the disclosure relates to synthetic materials comprising a twisted and interlaced fibril collagen matrix with a collagen density of greater than 600 micrograms per square centimeter and the collagen fibers maintain D-periodicity. In certain embodiments, the collagen fibers are separated on average by greater than 200 nanometers and less than 1 micrometer. In certain embodiments, the collagen matrix has a greater fibril alignment frequency in one direction. The material is typically in the form of a sheet with a thickness of less than 50 micrometers and has a continuous surface area of greater than 2 square centimeters.

In some embodiments, the thickness of the sheet is about 0.5 micrometer (μm) to 50 μm. For example, the thickness of the sheet may be about 0.5 μm to 45 μm, 0.5 μm to 40 μm, 0.5 μm to 35 μm, 0.5 μm to 30 μm, 0.5 μm to 25 μm, 0.5 μm to 20 μm, 1 μm to 45 μm, 1 μm to 40 μm, 1 μm to 35 μm, 1 μm to 30 μm, 1 μm to 25 or 1 μm to 20. In some embodiments, the thickness of the sheet is about 1 μm, 5 μm, 10 μm, 15 μm, 20 μm, 25 μm, 30 μm, 35 μm, 40 μm, or 45 μm.

In some embodiments, the continuous surface area of the sheet is about 2 square centimeters (cm²) to about 35 (cm²). For example, the continuous surface area of the sheet may be about 2 cm², 3 cm², 4 cm², 5 cm², 6 cm², 7 cm², 8 cm², 9 cm², 10 cm², 15 cm², 20 cm², 25 cm², 30 cm², or 35 cm².

In certain embodiments, the material further comprises an elastic polymer, e.g., elastin or elastin-like polymer comprising tetrapeptide, pentapeptide, or hexapeptide repeats comprising proline. In certain embodiments, the elastin or elastin-like polymer is layered on or infused into the collagen matrix. In certain embodiments, the elastic polymer comprises peptide repeats of [YaaPUaaXaaZaaₚ]ₙ (SEQ ID NO:1) wherein Yaa is glycine, alanine, lucine, isolucine, or valine; P is Pro; Uaa is glycine, alanine, lucine, isolucine, or valine; Xaa is aspartic acid, glutamic acid, glycine, alanine, lucine, isolucine, or valine, or any amino acid except Pro; Zaa is glycine, alanine, lucine, isolucine, or valine; p is 0, 1, 2, 3, 4, 5, or 6; and n is 1 to 1000, inclusive.

In certain embodiments, the elastic polymer comprises a protein copolymer comprising at least one hydrophilic block and at least one hydrophobic block, said copolymer having a first hydrophobic end block, a second hydrophobic end block, and a middle hydrophilic block (e.g., as described in Cappello J. Genetically engineered protein polymers. In: Domb A J, Kost J, Wiseman D M, editors. Handbook of Biodegradable Polymers. Amsterdam: Harwood; 1997. P. 387-414; Capello J, et al. Biotech. Progr. 1990; 6 (3)198-202; McGrath K P, et al. Protein-based materials. Boston: Birkauser; 1997; and McGrath K P, et al. Biotechnol. Progr. 1990; 6 (3):188-92, each of which is incorporated by reference herein).

Elastic polymers as provided herein may be recombinant elastin analogs. Such analogs, in some embodiments, provide a resilient matrix and a thromboresistant blood-contacting surface. Examples of recombinant elastin analogs for use as provided herein are described by Caves J M, et al. Biomaterials 2010; 31 (27):7175-82; Waterhouse A, et al., Tissue Eng. Part B Rev. 2011; 17 (2):93-9; and Jordan S W, et al. Biomaterials 2007; 28 (6):1191-7, each of which is incorporated by reference herein).

In certain embodiments, the disclosure relates to aligned fibrous collagen matrix fabricated by strain alignment of collagen gels and subsequent drying providing collagen fibril alignment over centimeter length scales that retain D-periodicity. In certain embodiments, fibrous materials are embedded in a recombinant elastin, or co-cast from mixtures of collagen and recombinant elastin, to form protein composite sheets. In certain embodiments, the sheets are rolled on a mandrel, and individual layers are reconstituted by thermal cooling and reheating.

Typically, the collagen layer, sheet, or mat is a matrix of continuous collagen fibers separated by less than about 1 micrometer on average having a collagen fiber of about 70 to about 90 nanometers, wherein the matrix fibers contain D-periodicity. In certain embodiments, the sheet is a thickness of less than about 100, 50, 40, 30, 20, or 10 micrometers, and has a surface area of greater than 1, 2, 3, 4, 5, 10, or 100 square centimeters. In certain embodiments, the sheet is a thickness of more than 30, 20, 10, 5, or 2 micrometers, and has a surface area of greater than 1, 2, 3, 4, 5, 10, or 100 square centimeters. In certain embodiments, the disclosure relates to collagen matrices having a spatial concentration of about or greater than 600, 700, or 800 μg/cm². In some embodiments, the collagen matrices have a spatial concentration of about 600 μg/cm² to about 1000 μg/cm², or about 600 μg/cm² to about 1500 μg/cm². In some embodiments, the concentration of collagen in the collagen matrices is about 0.5 mg/ml to about 8 mg/ml, about 1 mg/ml to about 5 mg/ml, or about 1.25 mg/ml to about 5 mg/ml. For example, the concentration of collagen in the collagen matrices may be about 0.5 mg/ml, 1 mg/ml, 1.25 mg/ml, 1.5 mg/ml, 1.75 mg/ml, 2 mg/ml, 2.25 mg/ml, 2.5 mg/ml, 2.75 mg/ml, 3 mg/ml, 3.25 mg/ml, 3.5, mg/ml, 3.75 mg/ml, 4 mg/ml, 4.25 mg/ml, 4.5 mg/ml, 4.75 mg/ml, 5 mg/ml, 5.25 mg/ml, 5.5 ml/ml, 5.75 mg/ml or 6 mg/ml.

In certain embodiments, the collagen matrix has a tensile strength of greater than 5 or 6 MPa. In certain embodiments, the collagen matrix has about the same fibril alignment frequency in any direction, e.g., about 3%. In certain embodiments, the material has a fibril alignment frequency of greater than or about 3%, 3.5%, or 4% in one direction. In certain embodiments, the matrix has between 8%, 7%, 6%, 5%, or 4% and 3% alignment frequency in one direction or in any direction.

In certain embodiments, the disclosure relates to strain aligned collagen matrix coated or crosslinked with a biodegradable material such as elastin-like proteins, PE (polyethylene), PTFE (polytetrafluoroethylene), PLGA (poly-lactic-co-glycolic acid), perfluoroalkoxy (PFA), fluorinated ethylene propylene (FEP), polycaprolactone, polyglycolide, polylactic acid and/or poly-3-hydroxybutyrate. Other elastin-like polymers are known in the art and may be used as provided herein.

A variety of crosslinking agents are known in the art and may be used herein. Examples of crosslinking agents include, without limitation, di(ethylene glycol)dimethacrylate, n,n′-(1,2-dihydroxyethylene)bisacrylamide, divinylbenzene, divinylbenzene, p-divinylbenzene, ethylene glycol diacrylate, ethylene glycol dimethacrylate, 1,6-hexanediol diacrylate, 4,4′-methylenebis(cyclohexyl isocyanate), 1,4-phenylenediacryloyl chloride, poly(ethylene glycol)diacrylate, poly(ethylene glycol)dimethacrylate, tetra(ethylene glycol)diacrylate, tetraethylene glycol, and triethylene glycol dimethacrylate.

In certain embodiments, the disclosure relates to materials comprising a) a collagen layer; and b) a first elastic polymer layer adjacent to the collagen layer comprising tetrapeptide, pentapeptide, or hexapeptide repeats comprising proline. In certain embodiments, the material further comprises a second elastic polymer layer adjacent to the collagen layer configured such that the collagen layer is a sheet between the first and second elastic layers. The first and/or second elastic polymer layers typically comprise peptide repeats of [YaaPUaaXaaZaaₚ]ₙ (SEQ ID NO:1), wherein Yaa is glycine, alanine, lucine, isolucine, or valine; P is Pro; Uaa is glycine, alanine, lucine, isolucine, or valine; Xaa is aspartic acid, glutamic acid, glycine, alanine, lucine, isolucine, or valine, or any amino acid except Pro; Zaa is glycine, alanine, lucine, isolucine, or valine; p is 0, 1, 2, 3, 4, 5, or 6; and n is 1 to 1000.

In certain embodiments, the first or second elastic polymer layer comprises a protein copolymer comprising at least one hydrophilic block and at least one hydrophobic block, said copolymer having a first hydrophobic end block, a second hydrophobic end block, and a middle hydrophilic block. In certain embodiments, the middle block comprises [YaaPUaaXaaZaaₚ]ₙ (SEQ ID NO:1), wherein Yaa is glycine, alanine, lucine, isolucine, or valine; P is Pro; Uaa is glycine, alanine, lucine, isolucine, or valine; Xaa is; aspartic acid, glutamic acid, glycine, alanine, lucine, isolucine, or valine, or any amino acid except Pro; Zaa is glycine, alanine, lucine, isolucine, or valine; p is 0, 1, 2, 3, 4, 5, or 6; and n is 1 to 1000.

In certain embodiments, the first and second end blocks comprise [YaaPUaaXaaZaaₚ]ₙ (SEQ ID NO:1), wherein Yaa is glycine, alanine, lucine, isolucine, or valine; P is Pro; Uaa is glycine, alanine, lucine, isolucine, or valine; Xaa is; glycine, alanine, lucine, isolucine, or valine; Zaa is glycine, alanine, lucine, isolucine, or valine; p is 0, 1, 2, 3, 4, 5, or 6; and n is 1 to 1000.

In certain embodiments, the middle block comprises [(VPGAG)ₚVPGXaaG(VPGAG)_{q}]ₙ (SEQ ID NO:2), wherein Xaa is glutamic acid, aspartic acid, arginine, histidine, lysine, serine, threonine, asparagine, or glutamine; p is 0, 1, 2, or 3; q is 0, 1, 2, or 3; n is 1 to 1000, inclusive, or n is between 10 and 100, inclusive. In certain embodiments, the middle block comprises (Val-Pro-Gly-Glu-Gly) (SEQ ID NO:4).

In certain embodiments, the first and second end blocks comprise IPAVG (SEQ ID NO:3) or [IPAVG]ₙ (SEQ ID NO:3) wherein n is 1 to 200, inclusive, or 5 to 200, inclusive.

In certain embodiments, the copolymer comprises a peptide sequence comprising lysine between the middle block and the first or second block.

In certain embodiments, the disclosure relates to methods of making a sheet of a collagen matrix comprising: a) mixing an acid solution comprising acid soluble collagen with a buffer under conditions such that a collagen gel forms; b) incubating the collagen gel in an aqueous buffer solution at a neutral pH for more than one day providing a cured layer of collagen; c) separating the cured layer of collagen from the buffer solution; and d) drying the cured layer of collagen to provide dried collagen. Typically, the collagen gel is stretched in the buffer solution to greater than or about 1%, 5%, 10%, or 20% of the original length in one direction providing a stretched layer of collagen stretched at a speed of less than or about 200, 100, 50, 10, or 5 micrometers per second, wherein the stretched layer of collagen is dried providing a stretched dried layer of collagen. In some embodiments, collagen as provided herein is natural collagen, while in other embodiments, collagen is synthetic or recombinant. Recombinant collagen may be produced using, for example, bacterial, insect or yeast cells. Collagen may be obtained from mammalian sources included, without limitation, human, bovine, porcine and the like. The collagen may by purified or partially purified. In some embodiments, the collagen is obtained by enzymatic digestion. In certain embodiments, solubilized collagen is obtained from rat tail tendon or calf skin or by enzymatic digestion of collagen. In some embodiments, the collagen is Type I collagen. In some embodiments, the collagen is Type II collagen.

In certain embodiments, the method further comprises the step of applying a layer of collagen gel to the stretched dried layer of collagen and drying the collagen gel under conditions such that a coated, stretched dried layer of collagen forms.

In certain embodiments, the method further comprises the steps of hydrating the stretched dried layer of collagen providing a hydrated stretched layer of collagen, applying a layer of collagen gel to the hydrated stretched layer of collagen and drying the hydrated stretched collagen gel under conditions such that a coated, stretched dried layer of collagen forms.

In certain embodiments, hydrating the stretched dried layer of collagen is performed on a cylindrical surface wherein the first stretched direction is parallel to the axis of the cylindrical surface.

In certain embodiments, the disclosure relates to methods of producing a material comprising a layer of collagen and a layer of elastic polymer comprising: a) cooling an acid solution to less than 15 degrees Celsius providing a cooled solution comprising, acid soluble collagen, and a protein comprising peptide repeats of [YaaPUaaXaaZaaₚ]ₙ (SEQ ID NO:1), wherein Yaa is glycine, alanine, lucine, isolucine, or valine; P is Pro; Uaa is glycine, alanine, lucine, isolucine, or valine; Xaa is aspartic acid, glutamic acid, glycine, alanine, lucine, isolucine, or valine, or any amino acid except Pro; Zaa is glycine, alanine, lucine, isolucine, or valine; p is 0, 1, 2, 3, 4, 5, or 6; and n is 1 to 1000, inclusive; b) neutralizing the cooled solution such that a collagen layer forms; and c) warming the solution under conditions such that an elastic layer forms.

In certain embodiments, the method further comprises the steps of removing the solution from the collagen and elastic layers; and drying the layers to provide a dried material with a collagen layer and an elastic layer.

In certain embodiments, the disclosure relates to methods of producing a material comprising: a) contacting a dried collagen sheet with a solution cooled to less than 15 degree Celsius wherein the cooled solution comprises a protein comprising peptide repeats of [YaaPUaaXaaZaaₚ]ₙ (SEQ ID NO:1) wherein Yaa is glycine, alanine, lucine, isolucine, or valine; P is Pro; Uaa is glycine, alanine, lucine, isolucine, or valine; Xaa is aspartic acid, glutamic acid, glycine, alanine, lucine, isolucine, or valine, or any amino acid except Pro; Zaa is glycine, alanine, lucine, isolucine, or valine; p is 0, 1, 2, 3, 4, 5, or 6; and n is 1 to 1000, inclusive; and b) warming the solution under conditions such that an elastic polymer layer forms over the dried collagen sheet.

In certain embodiments, the disclosure relates to a composition greater than about 30%, 40%, 45%, or 50% by weight collagen impregnated with an elastin-like protein. In certain embodiments, the spatial concentration of the material is about or greater than 1300, 1200, or 1000 μg/cm².

In certain embodiments, the disclosure relates to a method of making a collagen material impregnated with an elastin-like protein comprising mixing acid soluble collagen and elastin-like protein in a solution at below 14° C. and adding a buffer solution to the solution under conditions such that a collagen gel forms. In certain embodiments, the ratio of acid soluble collagen to elastin-like protein is about or greater than 1 to 1 by weight. In certain embodiments, the acid soluble collagen is at a concentration lower than 2.5 mg/ml.

In certain embodiments, the disclosure relates to materials made by the process disclosed herein. In certain embodiments, the disclosure relates to artificial vascular prosthesis comprising a material disclosed herein.

In certain embodiments, the disclosure relates to methods of producing patterns comprising cutting a material disclosed herein. The cutting is typically done by an excimer laser, carbon dioxide laser, or other tool. In certain embodiments, the pattern comprises a liner or nonlinear pattern or holes.

In certain embodiments, the disclosure relates to materials disclosed herein, such as described collagen matrices optionally containing elastin-like proteins, comprising one or more cells. In some embodiments the collagen matrices comprises embryonic or adults stem cells (e.g., pluripotent or induced pluripotent stem cells) or progenitor cells. The cells may be, for example, fibroblast cells (e.g., dermal fibroblast cells), epithelial cells, mesenchymal, smooth muscle cells, and bone cells. Other examples of cells for used as provided herein include, without limitation, mesenchymal stem cells, epithelial progenitor cells, and endothelial progenitor like-cell fibroblasts.

In certain embodiments, the disclosure relates to materials disclosed herein comprising a therapeutic agent such as an anti-inflammatory agent, anticoagulant, or antibiotic. An anti-inflammatory agents refers to a substance that reduces inflammation. Examples of anti-inflammatory agents for use as provided herein include, without limitation, steroids and non-steroidal anti-inflammatory drugs such as aspirin, ibuprofen, and naproxen. Anticoagulants prevent coagulation (or clotting) of blood. Examples of anticoagulants include, without limitation, heparin, anti-thrombin III, fibrin, anti-thromboplastin, heparan sulphate, protein C, protein S, coumarins, and heparin (including heparin derivatives). Antibiotics include antibacterial, antimicrobial, and antifungal agents that inhibits growth of the respective organism. Examples of antibiotics for use as provided herein include, without limitation, penicillins, cephalosporins, and carbapenems.

In certain embodiments, the disclosure relates to materials disclosed herein comprising bone granules or minerals, calcium phosphates, hydroxyapatite, tricalcium phosphate, or calcium sulphate.

In certain embodiment, the disclosure relates to cellularized vascular graft composites optionally comprising an ablated pattern. Collagen-elastin like materials are seeded with cells, e.g., bone marrow-derived stem cells, cultured, and formed into a cellularized vascular graft. Other contemplated cells include endothelial progenitor cells and mesenchymal stem cells, umbilical cord cells, and peritoneal cells. In certain embodiments, the grafts are seeded with smooth muscle cells.

In certain embodiments, the disclosure relates to vascular graft compositions of collagen-elastin like materials comprised cell homing compounds conjugated to the material. In certain embodiments, CD34 antibody, CD31 antibody, and/or SDF-1 are conjugated to the surface of the material.

In certain embodiments, the disclosure relates to functionalization of cell binding/cell homing sequences to labile groups on collagen or elastin, e.g., conjugation through lysine residues. In another embodiment, a CD34 antibody is used to home and conjugate circulating endothelial progenitor cells to graft surfaces. In certain embodiments, the materials are conjugated with an antithrombotic such as those selected from thrombomodulin, warfarin, acenocoumarol, phenprocoumon, atromentin, phenindione, heparin, fondaparinux, idraparinux, rivaroxaban, apixaban, hirudin, lepirudin, bivalirudin, argatroban, and dabigatran to reduce luminal thrombosis.

In certain embodiments, the disclosure relates to methods of soft tissue repair and replacement using patches made from materials disclosed herein. In certain embodiments, the disclosure relates to methods of abdominal wall and hernia repair using patches made from materials disclosed herein. In certain embodiments, the disclosure relates to methods of vascular tissue replacement, artificial vascular grafts, and vascular patches using materials disclosed herein. In certain embodiments, the disclosure relates to artificial tissue such as artificial skin, a matrix for muscle regeneration, dura mater, pelvic floor, cartilage, and bone, as well as cardiac patches, optionally for drug and/or cell delivery comprising materials disclosed herein.

In certain embodiments, the disclosure contemplates using different gelation systems, temperatures and mechanisms, e.g., freeze drying to induce long crystal formation and structural anisotropy.

In certain embodiments, the disclosure contemplates the incorporation of calcites and other apatites to enhance the hardness of materials disclosed herein, e.g., co-casting with mineralized hydroxyapatite and calcium phosphates.

In certain embodiments, the disclosure contemplates materials co-cast with proteoglycans and glycoaminoglycans to generate hydrophilic matrices that coordinate water molecule.

In certain embodiments, the disclosure relates to ablation patterns for creating a variety of artificial tissues, e.g., hierarchical blood vessels, a lung diffusion barrier between alveoli and epithelial cells, blood brain barrier replacement, as a soft tissue scaffold with cardiomyocyte growth, localized seeding of cells for liver lobule, pancreas, and kidney nephron functional unit regeneration.

In certain embodiments, the disclosure relates to aligning muscle cells on collagen by ablation of linear lines on collagen materials disclosed herein.

In certain embodiments, the disclosure contemplates materials disclosed herein such as collagen and elastin IPN matrices that are conjugated or trap with therapeutics and small proteins that have labile crosslinking groups, e.g., small molecules, or nano- or microparticles physically embedded in the collagen or IPN structure or tethered to collagen or IPN matrix for use in graft-tissue response modulation or localized drug delivery.

### BRIEF DESCRIPTION OF THE FIGURES

FIG. 1 schematically illustrates of the mechanical setup used to induce strain alignment of collagen gels. Collagen gels were cast in a rectangular mold (Length×Width×Thickness: 100×80×4 mm) at 4° C. (A), incubated in a fibril incubation buffer for 48 h at 37° C. (B), mounted on a motorized stretcher (C), and stretched to a strain of 0%, 10% or 20% stretch, at 3 μm/s or 300 μm/s (D).

FIG. 2 shows scanning electron micrographs of the ultrastructure of collagen matrices. (A) isotropic microstructure (scale bar: 1 μm), (B) with 83.1±9.44 nm fibrils (scale bar: 200 nm). Transmission electron micrographs showing (C) a dense fiber matrix (scale bar: 1 μm) and (D) native collagen banding showing preservation of the native D-periodic banding pattern (scale bar: 200 nm).

FIG. 3 shows SEM images and histograms of different stretching rate, strain amount, and concentration dependence on alignment of collagen matrices. Top panel shows SEM images, and bottom panel shows histograms of FFT analyses of SEM images of 9 regions of 4 independent samples, of (A & D) 2.5 mg/mL matrix aligned at 300 μm/s to 10% strain, (B & E) 2.5 mg/mL matrix aligned at 3 μm/s to 10% strain and (C & F) 2.5 mg/mL matrix aligned to 20% strain. Scale bar: 500 nm.

FIG. 4 shows data indicating the alignment of collagen fibrils based on Gaussian fit of alignment data derived from FFT of SEM images at 10 k× magnification. (A) Maximum relative frequency of fibrils, (B) Full width at half maximum, FWHM (where majority of fibrils reside).

FIG. 5 shows data on the mechanical properties of 20% stretch aligned, 20% stretch aligned tested perpendicular to alignment, 10% stretch aligned, and (0%) unaligned 2.5 mg/mL collagen mats. (A) Tensile strength, (B) Strain at failure and (C) Young's modulus, (D) Fibril diameter and (E) Mat thickness. (A) Tensile strength and (C) Young's moduli depend on percent alignment of matrices. (B) Strain to failure, (D) Fibril diameter and (E) Mat thicknesses are similar for all constructs.

FIG. 6 shows data of a stress-strain plot of collagen matrices. Matrices were stretch-aligned to different amounts at a rate of 3 μm/s ( - - - : 20%, • • • • • • : 10%, - - - - :0%). Samples were cut into 20 mm long×5 mm wide strips and mechanically tested. Samples were pre-conditioned 15 times to 66% of failure strain, and then tested to failure.

FIG. 7 schematically illustrates embodiments of different fabrication strategies for layered collagen elastin nanocomposites. Collagen gels were cast at 4 mm thickness. (A) Collagen mats are dried to a dry thickness of approximately 10 μm, (D) and layered into multi-layer collagen mats. (B) Single layer collagen mats are embedded in elastin into a single ply, in a sandwich molding technique, (E) multi-layer mats are embedded into a multi-layer single ply composite. (C) single layer single ply composites are stacked into single layer multi-ply composites, (F) multi-layer single ply composites are stacked into multi-layer multi-ply composites.

FIG. 8 shows data on the mechanical properties of genipin crosslinked collagen mats. Increasing concentration of initial gels results in improved strength and stiffness with a commensurate increase in thickness (A,B,G,H). Increasing the number of layers shows an increase in strength and stiffness (C,D,I,J). Increasing initial thickness of 2.5 mg/mL collagen gels in 4 layer mats systems resulted in significantly stronger matrices (E,F,K,L).

FIG. 9 shows certain ablation schemes of collagen matrices. (A) Schematic of collagen mat ablated using an excimer laser to create a defined “wavy” collagen mat with linear supports, inset shows additional nomenclature. (B) Uniformity and transfer of wavy ablated pattern with high fidelity onto collagen mats, scale bar 500 μm. (C-D) Ablated collagen mat displayed clear excimer laser cuts with no apparent material damage, scale bar 100 μm.

FIG. 10 shows meso- and ultra-structure of collagen matrices of varying vertical strip width. (A-E) Optical micrographs of stainless steel (B) and aluminum-on-quartz masks (A, C, D, E) for Designs 1-5 (A-E), respectively, scale bar 500 μm. (F-J) Optical micrographs of genipin crosslinked collagen mats for Designs 1-5, respectively, scale bar 500 μm. SEM of wavy collagen matrices (K) 200×, wave edge (L) 5 k×, and magnified view of fibrillar structure (M) 50 k×, scale bars 300 μm, 10 μm, 1 μm respectively. TEM images of wavy collagen mats, (N) 10 k× bulk and (O) 10 k× wave edge, scale bar 1 μm.

FIG. 11 shows data on mechanical strength of ablated collagen mats, Designs 1-5. (A) Ultimate tensile strength of ablated crosslinked collagen mats. (B) Strain at failure of ablated collagen mats. (C) Young's modulus of ablated collagen samples.

FIG. 12 shows data on endothermic heat transitions of collagen matrices. Microdifferential scanning calorimetry of lyophilized collagen (solid), collagen mat (dotted), excimer ablated collagen film (dash), genipin crosslinked collagen mat (dash-dot). (n=3)

FIG. 13 shows cellularization of unablated and ablated scaffolds. (A) Unablated scaffold seeded with rMSCs at 100,000 cells/cm2, for 24 h Live (green)/Dead(red) stained. (B) Ablated scaffold seeded with rMSCs at 100,000 cells/cm2, for 24 h (Design 2). (C) Actin cytoskeletal staining and DAPI nuclei staining showing alignment of cells on ablated scaffolds.

FIG. 14 schematically illustrates the fabrication of acellular and cellularized grafts. (A) Collagen-elastin IPN mats are dried from collagen-elastin gels into defined thicknesses. (B) Mats can be cellularized with rMSCs at 100,000 cells/cm². (C) Acellular and cellularized mats are embedded in elastin at defined thicknesses dictated by plastic shims (purple). (D) Acellular and cellularized composite sheets are rolled on a mandrel to create vascular grafts.

FIG. 15 shows data on the mechanical properties of genipin crosslinked IPNs. Introducing elastin into collagen gels resulted in an improvement in strength and stiffness (A,B,G,H). Increasing collagen concentration while maintaining elastin concentration resulted in weaker matrices (C,D,I,J). Increasing the number of layers of a 1.25 mg/ml collagen, 1.25 mg/ml elastin IPN shows an unexpected improvement in strength and stiffness, indicating interpenetration of matrices and reinforcement (E,F,K,L).

FIG. 16 shows representative stress-strain plots showing the mechanical characterization of uncrosslinked 2.5 mg/ml collagen, 2.5 mg/ml elastin IPN. (A) Preconditioning curves of 2.5 mg/ml collagen-2.5 mg/ml elastin IPN, (black dot): first cycle, (open dot): 15ₜₕ cycle. (B) Characteristic mechanical response of IPN ( --- ), LysB10 ( - - - ) and 2.5 mg/ml collagen only matrix (dotted line), showing increase in stiffness and strain to failure with incorporation of elastin.

FIG. 17 shows meso- and ultrastructure of IPN grafts. (A) Photo of unimplanted graft segment, (B) long graft segment showing kink resistance, (C) Van Geison stained cross section of graft wall clearly delineating layers: collagen in IPN stained red, elastin yellow, scale bar 100 μm, (D) SEM of graft cross-section, scale bar 500 μm, (E) SEM of graft wall showing contiguous elastin layer and site of rolling initiation, scale bar 100 μm, (F) SEM of elastin structure on lumen of graft, scale bar 1 μm, (G) SEM of fibrous structure of 2.5 mg/ml collagen only mat showing fibrillar collagen, 50 k×, scale bar 1 μm, 10 k× inset showing global nanofibrous morphology, scale bar 2 μm, (H) SEM of fibrous structure of 2.5 mg/ml collagen, 2.5 mg/ml elastin IPN mat showing fibrillar collagen “decorated” with elastin, 50 k×, scale bar 1 μm, 10 k× inset showing global nanofibrous morphology, scale bar 2 μm, (I) SEM of nanofibrous region of graft impregnated with elastin, scale bar 1 μm. (J) TEM of IPN mat showing characteristic collagen banding, scale bar 1 μm. (K) and (L) TEM of cross-section of elastin (top) embedded IPN showing preservation of native structure, scale bar 10 μm and 0.5 μm respectively.

FIG. 18 shows cellularization of IPN. (A-I) Planar IPN seeded with rMSCs at 50,000, 100,000 and 200,000 cells/cm², showing cell adhesion (4 h-12 h) and spreading (12 h-24 h).

FIG. 19 shows Live/Dead staining for cell viability on graft surfaces. (A) Planar IPN seeded with rMSCs at 100,000 cells/cm² for 24 h embedded in elastin. (B) Cellularized composite imaged after 3 days. A series of 0.9 mm grafts rolled either with infused superficial elastin facing the lumen or infused IPN facing the lumen (E-F) were constructed. (C) rMSCs were seeded on adventitial side (IPN exposed) of rolled grafts at 100,000 cells/cm² for 24 h. (D) No cells present on luminal elastin side. (E) rMSCs were seeded on luminal side (IPN exposed) of rolled grafts at 100,000 cells/cm² for 24 h, (F) murine dermal microvascular ECs were seeded on luminal side (IPN exposed) of rolled grafts at 100,000 cells/cm² for 24 h. Scale bar is 300 μm.

FIG. 20 shows implantation of a 1 cm long 1.3 mm ID aortic interposition graft in the infrarenal suprailiac position. Gross morphology of the graft was noted: (A) Photo of graft at implant showing reddish color of blood flow, (B) Photo of graft at explant after exsanguination and perfusion fixation. (C) Evaluation of patency of lumen using contrast based angiographic computed tomography, * delineate graft.

FIG. 21 shows graft morphology and cellular infiltrate. (A) Masson's Trichrome staining of ECM of graft sections showing IPN layers in blue (Lys-B10 does not stain positively), and neointima, scale bar 100 μm, (B) magnified image showing trapped red blood cells and blue staining of neo-collagen matrix in neointima, (C) thin layer of mononuclear cells adherent on adventitial IPN surface. * indicates luminal side.

FIG. 22 shows an abdominal wall model and repair. An incisional hernia model was created by cutting through the abdominal wall to the peritoneum (A). A 12-layered collagen patch (B) or 1 mm thick control patch (F) was sewn into place using a 6-0 suture. (E) Representative image showing none of the treatment or control groups exhibits gross ventral re-herniation at any explant time points; bracket indicates approximate implant site. Un-degraded multilayer collagen (C) and Permacol™ (G) are clearly present at 1 month. Both collagen and control (G) are clearly present at 1 month. At 3 months, the collagen patch shows appreciable reintegration with host tissue (D) relative to control (H). Scale bar minor units in mm.

FIG. 23 shows extracellular matrix staining (Masson's Trichrome) of unimplanted and implanted samples. Collagen (A) and control (E) patches prior to implant show uniform thickness and distinct morphology. For collagen implants, wavy morphology collagen (arrows) is noted above muscle, and adjoining highly cellularized peritoneal layer at 1 month (B) is clearly delineated in the center of the recellularized implant at 2 months (C) and is seen in isolated pockets at 3 months (D). Control implants can be clearly distinguished from host tissue at 1 month (F), 2 months (G), and 3 months (H), resembling pre-implant structure and morphology. Scale bar A-D: 200 μm and E-H: 500 μm.

FIG. 24 shows staining of cellular infiltrate of implanted samples. Anti-rat vWF staining characterized endothelialization of 1 month collagen (A), 3 month collagen (B), 1 month Permacol™ (E), and 3 month Permacol™ (F) samples. Arrows point to circular vessel like structures. Monocyte/macrophage marker CD68 staining of 1 month collagen (C), 3 month collagen (D), 1 month Permacol™ (G) and 3 month Permacol™ (H). Total number of CD68⁺ nuclei decreases more in collagen implants compared to Permacol™ implants at 3 months. Scale bar 100 μm.

### DETAILED DESCRIPTION OF CERTAIN EMBODIMENTS

This disclosure relates to materials fabricated from collagen. In certain embodiments, the disclosure relates to materials comprising dehydrated stretched collagen matrices that retain D-periodicity and high density. In certain embodiments, the collagen materials are coated with an elastin-like protein polymer through direct contact with or without crosslinking agents. In certain embodiments, these materials are used in tissue repair (e.g., hernia repair). In other embodiments, these materials are arranged into cylinders and used as prosthetic vascular grafts.

Desirable attributes for fabricating and using artificial tissue scaffolds include: (1) minimal processing that allows for scalable manufacture, (2) a hierarchical structure that can be tailored both structurally and mechanically to match native tissue, (3) a conductive environment for cellular adhesion, growth, and proliferation, (4) degradation to yield non-toxic products, and (5) prevention of inflammatory responses. In certain embodiments, the collagen matrices provide herein have one or more of these properties.

In certain embodiments, the material has a sufficient burst pressure to prevent failure of the vessel and long-term fatigue resistance, a suitable compliance that approximates that of the vessel to prevent mechanical mismatch, and a strong enough suture retention strength to permit implantation and tolerate hydrodynamic and mechanical forces.

In some embodiments, collagen matrices as provided herein have an ultimate tensile strength (UTS) of about 0.5 to about 1.5 MPa. For example, the UTS of a collagen matrix may be about 0.5 MPa, 0.6 MPa, 0.7 MPa, 0.8 MPa, 0.8 MPa, 0.9 MPa, 1.0 MPa, 1.1 MPa, 1.2 MPa, 1.3 MPa, 1.4 MPa, or 1.5 MPa. In some embodiments, collagen matrices as provided herein have aUTS of 0.71±0.06 MPa, or about 0.7±0.1. In some embodiments, collagen matrices as provided herein have aUTS of 0.60±0.09 MPa, or about 0.60±0.1 MPa.

In some embodiments, collagen matrices as provided herein exhibit strain to failure of about 30% to about 40%. For example, the strain to failure of a collagen matrix may be about 30%, 31%, 32%, 33%, 34%, 35%, 36%, 37%, 38%, 39%, or 40. In some embodiments, collagen matrices as provided herein exhibit strain to failure of 37.1±2.2%, or about 37.0±2.5%. In some embodiments, collagen matrices as provided herein exhibit strain to failure of 38.5±4.5%, or about 38%±5%.

In some embodiments, collagen matrices as provided herein have a Young's modulus of about 1.5 MPa to about 2.5 MPa. For example, the Young's modulus of a collagen matrix may be about 1.5 MPa, 1.6 MPa, 1.7 MPa, 1.8 MPa, 1.9 MPa, 2.0 MPa, 2.1 MPa, 2.2 MPa, 2.3 MPa, 2.4 MPa, or 2.5 MPa. In some embodiments, collagen matrices as provided herein have a Young's modulus of 2.09±0.21 MPa, or about 2.0±0.5 MPa. In some embodiments, collagen matrices as provided herein have a Young's modulus of 1.55±0.38 MPa, or about 1.5±0.5 MPa.

In some embodiments, the resilience (e.g., a measure of recovered energy during unloading of matrices) of the collagen matrices may be about 50% to about 60%. That is, the specified percentage of energy may be recovered during loading and unloading cycles. For example, the resilience of the collagen matrices may be about 50%, 51%, 52%, 53%, 54%, 55%, 56%, 57%, 58%, 59%, or 60%. In some embodiments, the resilience of a collagen matrix may be about 58.9±4.4%, or about 59±5%.

In some embodiments, collagen matrices as provided herein have a compliance of about 1%/100 mmHg to about 5%/100 mmHg. For example, the compliance of a collagen matrix may be about 1%/100 mmHg, 1.5%/100 mmHg, 2%/100 mmHg, 2.5%/100 mmHg, 3%/100 mmHg, 3.5%/100 mmHg, 4%/100 mmHg, 4.5%/100 mmHg, or 5%/100 mmHg. In some embodiments, collagen matrices as provided herein have a compliance of 2.09±2.7±0.3%/100 mmHg, or about 2.5±0.5%/100 mmHg.

In some embodiments, collagen matrices as provided herein have a burst pressure of about 650 mmHg to about 1000 mmMg. For example, the burst pressure of a collagen matrix may be about 650 mmHg, 660 mmHg, 670 mmHg, 680 mmHg, 690 mmHg, 700 mmHg, 710 mmHg, 720 mmHg, 730 mmHg, 740 mmHg, 750 mmHg, 760 mmHg, 770 mmHg, 780 mmHg, 790 mmHg, 800 mmHg, 810 mmHg, 820 mmHg, 830 mmHg, 840 mmHg, 850 mmHg, 860 mmHg, 870 mmHg, 880 mmHg, 890 mmHg, 900 mmHg, 910 mmHg, 920 mmHg, 930 mmHg, 940 mmHg, 950 mmHg, 960 mmHg, 970 mmHg, 980 mmHg, 990 mmHg, or 1000 mmHg. In some embodiments, collagen matrices as provided herein have a compliance of 830±131 mmHg, or about 830±150 mmHg. In some embodiments, vascular grafts as provided herein may exhibit burst pressures that are about threefold to about fourfold higher than maximum physiological pressures (Kumar V A, et al., Cardiovasc. Eng. Technol. 2011; 2 (3):137-48, incorporated herein by reference).

In certain embodiments, the material has a non-fouling surface to prevent thrombosis and to prevent unwanted activation of the innate immune response.

To this end, in certain embodiments, the disclosure relates to collagen fiber-elastin reinforced nanocomposites for utility in tissue and vascular graft applications. Certain materials show the ability to be mechanically tailored to match a variety of tissue based substrates by the variation of initial collagen and elastin concentrations. An unexpected increase in mechanical strength (UTS) and stiffness (Young's Modulus) is shown as a function of network interpenetration and densification through matrix layering. To further modulate mechanics, genipin crosslinking was used. Through the use of dense collagen-elastin interpenetrating networks (IPNs) embedded with recombinantly expressed elastin in a sandwich molding process, the ability to rapidly create rolled tubular constructs that exhibit mechanical properties similar to native tissue was demonstrated. The native ultrastructure of collagen is preserved with the addition of elastin in IPNs as well as the cellular adhesiveness. IPN matrices show rapid cellular adhesion, spreading and proliferation at modest cell densities (100,000 cells/cm²). Embedding cell seeded constructs with elastin and subsequent rolling allows for the formation of tubular cellularized composites. Implanted vascular grafts show excellent stability and lack of neointimal hyperplasia, aneurysmal dilation, or luminal thrombosis/stenosis.

### Terms

The term “collagen” refers to any of the fibril forming collagen proteins derived from natural sources or synthetically prepared proteins comprising tripeptide repeats of the amino acids Glycine-Proline-Hydroxyproline. Proline and hydroxyproline may be substituted with other amino acids; however, proline and hydroxyproline are the most abundant amino acids in those positions. Collagen further forms a coiled structure that leads to the formation of fibrils. It is contemplated that certain collagen proteins comprise some lysine substitutions for proline and hydroxyproline. Crosslinking agents typically form a covalent bridge between lysine residues. A threshold number of lysine residues allow for water solubility in acidic conditions varying on the acidity of the solution and the extent of lysine substitution.

The term “D periodicity” refers to characteristic banded that appears when viewing collagen fibrils, i.e., a regular transverse banding with axial periodicity D, where D is around 60-90 nm. D has been reported to be 67 nm in rat tail tendon collagen. Different values have been reported in other tissues such as skin. Dehydration typically leads to lower values of D. Tzaphlidou, Micron, 2001 32:337-339, describes a method of measuring axial periodicity of collagen. This reference is hereby incorporated by reference.

The term “tensile strength (TS)” or “ultimate tensile strength (UTS)” refers to the maximum stress that a material can withstand while being stretched before necking, i.e., when a cross-section of the material starts to significantly contract. Tensile strength is typically measured as force per unit area. A pascal is the number of newtons per square meter (N/m²).

### Fabrication and Characterization of Large Scale Structurally and Mechanically Anisotropic Nanofibrous Collagen Matrices

Collagen based fabrics can be aligned by stretching to generate structural and mechanical anisotropy. Collagen gels are generated by the neutralization of acidified Type I monomeric rat tail tendon collagen in a phosphate based buffer. Gel dimensions are dependent on the volume of collagen solution and buffer used, and allow for large structures to be fabricated. Fibrillogenesis within gels is further enhanced by incubation in a fiber incubation buffer. Gels are subsequently dried to less than 1% of their original thickness to create high density collagen mats, 4 mm cast gel dried to 28 μm. Structural anisotropy was generated by adhering gels onto mechanical supports and stretching at rates of 3 μm/s and 300 μm/s to strains of 10% and 20%. Previous reports of gelation systems and fabrication of smaller-scale anisotropic collagen matrices have been limited in size (sub-micron to millimeter scale) which have shown lack of scalability or utility for regeneration of large tissue replacements.

Dependence of gelation conditions on ultrastructure of collagen gels. Collagen gelation kinetics is highly dependent on collagen isolation method, initial collagen concentration, temperature of gelation, pH and presence of ions. Pepsin digested collagen structures are devoid of telopeptide sequences that are important to fibril formation with recapitulation of native collagen D-periodic structure, unlike acid solubilized collagen which still retains telopeptide sequences. The literature is replete with conflicting reports on fibril diameter and parameters that influence gelation. Reports have noted the effect of longer gelation times, and lower initial concentrations of collagen allow more time for fibrillogenesis without spatial restrictions from adjacent fibrils. However, studies herein indicate little difference in fibril diameter as a function of concentration (0.3125 mg/mL-2.5 mg/mL) in our gelation conditions, buffers used, stretch rate or stretch amount. See Table 1.

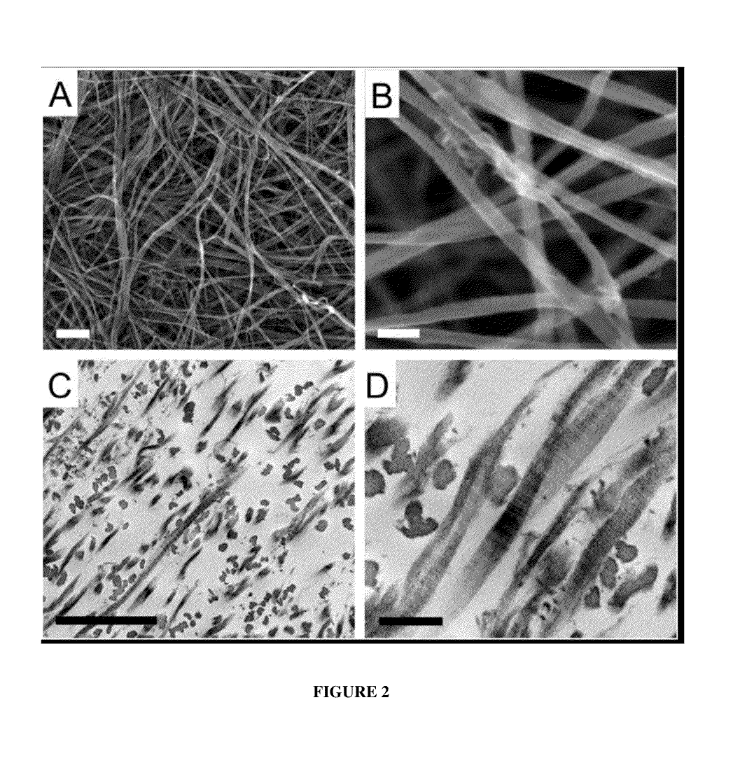

These small nanoscale differences do not directly translate to larger scale mechanical differences in ultimate tensile strength or strain at failure of centimeter scale constructs. Rather, there is a dependence on processing conditions and architectural arrangement of collagen fibrils in terms of alignment and packing density. Further the importance of D periodicity is exemplified by the characteristic 67 nm banding pattern of collagen (FIG. 2 C-D), which helps maintain the native structure of the collagen. This is thought to be important in higher order architectures that involve fibrillar collagen formation and preservation of cell binding moieties (ex. GFOGER (SEQ ID NO: 5) which mediates binding with cell surface integrins).

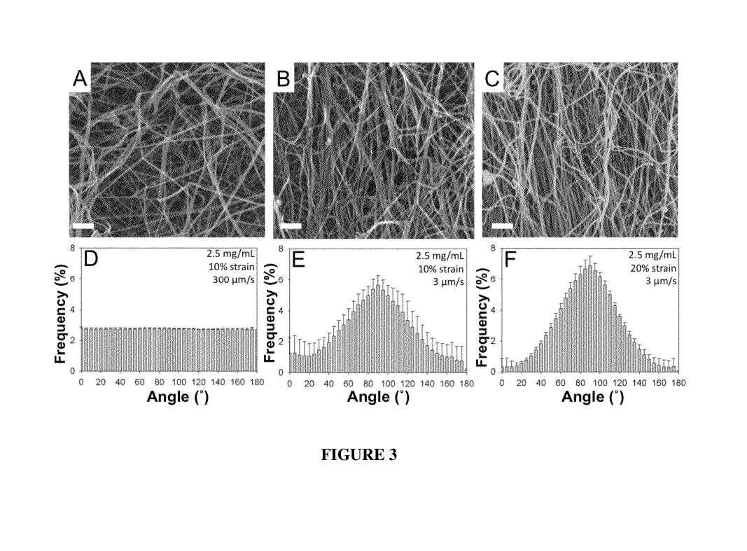

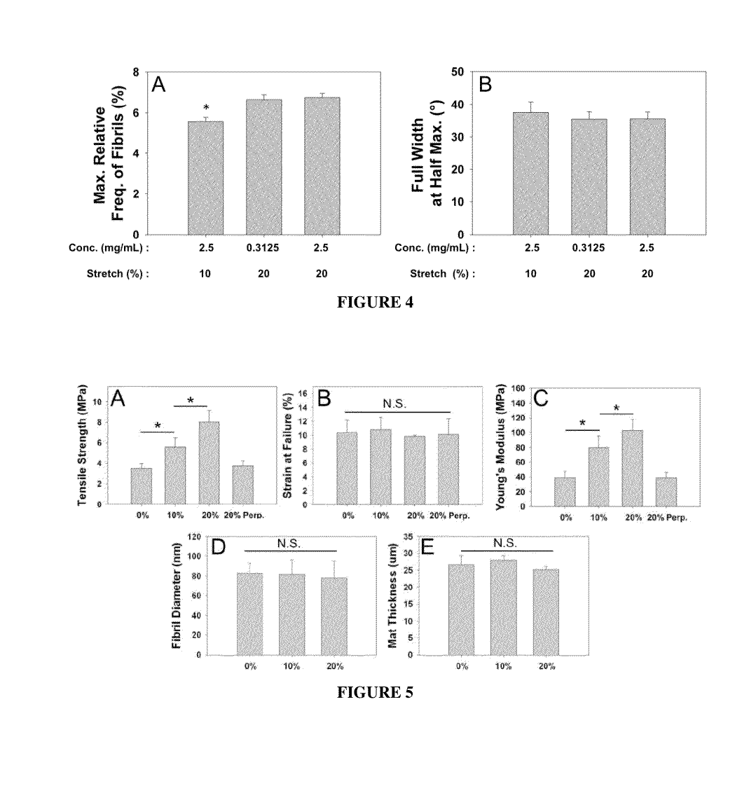

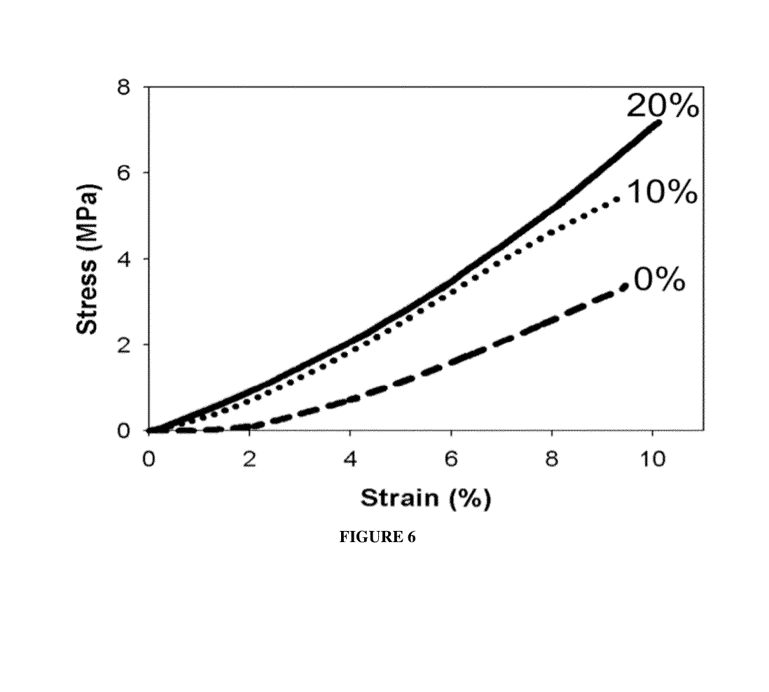

Generation of structural anisotropy within fiber matrices has been known to significantly improve their strength. Specific to collagen, our group and others have shown that anisotropic collagen structures can withstand greater mechanical load bearing applied in the direction of fibrils. Stretching of collagen gels has been shown not only to yield structural anisotropy and linear alignment of collagen fibrils in the direction of applied strain but also mechanical strengthening and reinforcement. See Table 1 and FIGS. 3 and 4. Although of significant strength, uncrosslinked collagen constructs may degrade more quickly and are of lower strength than crosslinked constructs. Therefore, a crosslinking scheme was employed to strengthen our collagen matrices and modulate potential degradation. Genipin, a naturally occurring crosslinker, known specifically for its ability to conjugate lysine residues and impart significant strength onto bioengineered matrices, has also been established to be biocompatible. Genipin crosslinked matrices exhibit an increase in ultimate tensile strength and stiffness, Young's modulus, with little to no change in strain at failure. Ultimately, uncrosslinked and crosslinked collagen constructs allow for the generation of a variety of mechanically tunable structures which can be adapted to several tissue engineering applications, including the development of blood vessels, cartilage, tendon, abdominal wall defect replacements or artificial skin.

### Use of Purified Collagen and Recombinantly Expressed Elastin to Mimic the Extracellular Matrix

While collagen has excellent cell adhesive properties, for applications that involve contact with blood, collagen is known to be thrombogenic. As such, we have developed a sandwich molding technique to infuse recombinantly expressed elastin into collagen matrices. Triblock co-polymer elastin analogs have shown the ability to undergo an inverse temperature phase transition in aqueous solutions. Of specific mention is Lys-B10, the elastin analogue used in this study. The hydrophobic (Ile-Pro-Ala-Val-Gly) (SEQ ID NO:3) block, flank a central hydrophilic midblock (Val-Pro-Gly-Glu-Gly) (SEQ ID NO:4) that aids in co-ordination of water molecules in aqueous solutions. However, above the lower critical solution temperature, the hydrophobic endblocks co-accervate, yielding a hydrogel. By introducing crosslinkable moieties into the elastin structure to promote intra/inter molecular crosslinking, one can crosslink co-elastin-like polymers to other protein based materials or compatible substrates through the aid of labile lysine residues. Further, given the inverse transition temperature, one is able to utilize elastin as a “glue” to adhere layers of collagen-elastin composites together. This further results in multi-ply composites, formed from single layer or multi-layer collagen mats infused with elastin. Consequently a series of thick composites were created that can be used for soft tissue repair and replacement and has utility in blood contacting applications.

Tunable ECM Mimetics with Enhanced Mechanical Properties

Increasing collagen concentration resulted in increased strength and stiffness for gels cast at the same thickness (4 mm). This is a direct result of greater amounts of protein present in the mats. Lower concentration mats potentially have micro-inhomogenieties or flaws which are masked when absolute protein amount increases, which, however, did not have an effect on strain to failure. Increasing the number of layers of collagen resulted in an increase in the ultimate tensile strength (UTS) of collagen matrices. This is a surprising discovery as UTS is the force normalized to the cross-sectional area. As such, the strength and stiffness is expected to remain constant. However, interestingly, there appears to be structural reinforcement of collagen matrices when they are layered. It may be that there is integration of the layers with each other, which potentially results in buttressing of fibrillar microstructure. During mechanical testing, failure of matrices occurred through transverse fracture in the direction perpendicular to axial stretch without delamination of collagen layers. Additionally, layered structures were thinner than multiple single layers, further suggesting collagen fibrils between layers were integrating between mats, and potentially generating a compressed randomly interwoven structure.

One variable altered to modulate mechanical properties of collagen mats was initial gel thickness. Initial collagen gel thickness variation resulted in an increasing amount of strength of matrices. Again, although normalized by thickness, the expected strength and stiffness should remain the same. However, higher initial gel thicknesses resulted in stronger gels that dried to stronger mats. Thicker gels have greater packing and compaction exhibiting a non-linear increase in thickness with increased initial collagen gel thickness. 8 mm and thicker gels were not as stable and tended to shear parallel to the plane of casting when removed from the mold.

### Optimization of a Protein-Based Laser Ablation Strategy and Preservation of Native Protein Structure

The primary advantage of excimer laser use is that ablation of tissue materials takes place with minimal damage to the surroundings. The benefit of excimer laser ablation is that it excites the molecular bonds sufficiently to dissociate them, ablate them, without thermal decomposition to elemental compounds. Further, the dissociated molecular products are cleared by an airstream which leaves a “clean” and non-denatured substrata that maintains native phenotype. Although non-thermal in nature, excimer laser ablation and other UV based optical ablation schemes generate small amounts of localized heat when maintained on a particular locus.

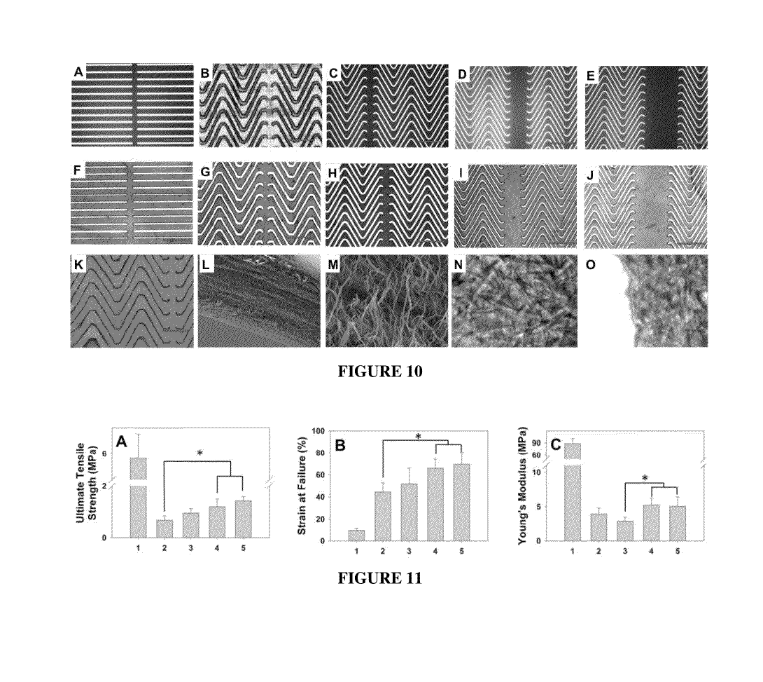

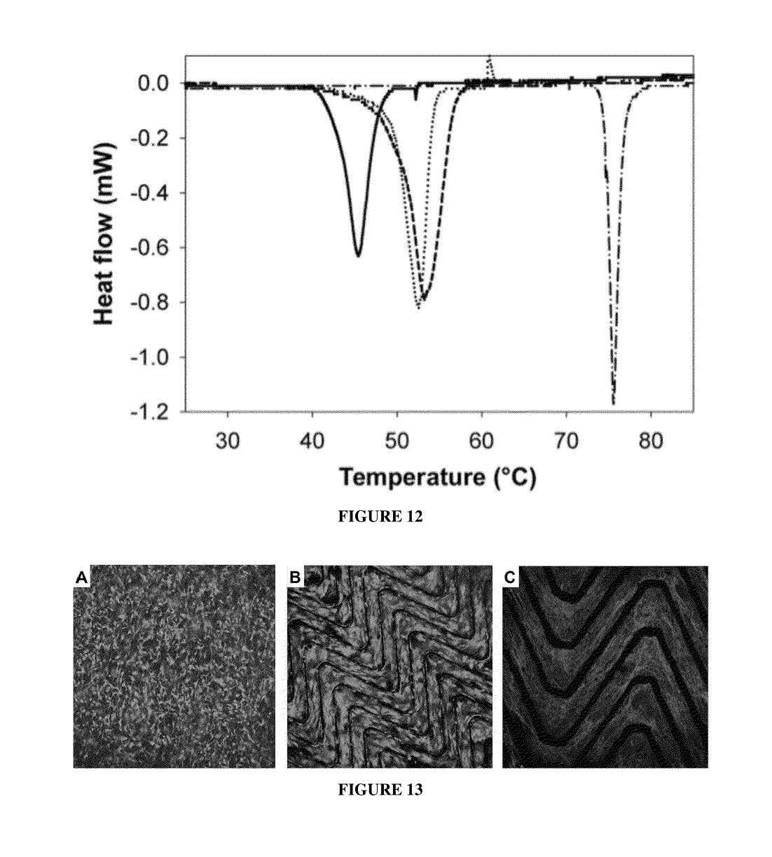

Studies were done to determine the optimal conditions that result in collagen ablation without denaturation. Parameters such as fluence (spatial laser energy density) and rastering of the substrate, with multiple passes over the same region ensured collagen matrices were ablated with minimal thermal denaturation as demonstrated by ultrastructural analysis and differential scanning calirometry. Conventional laser ablation can be achieved in two primary modes—direct-write or rastering (over a mask). The former is typically more time intensive and involves “writing” a pattern of individual features on the substrate. The latter, however, involves moving a relatively larger laser spot across the substrate. When coupled with a mask that is laser opaque, features, as determined by the mask, are ablated. Although resolution of excimer laser ablation was of magnitude 2-10 μm. Metal and aluminum-on-quartz masks were generated that attenuate UV light, but allow transmission in 10 μm or greater gaps (features). Consequently, one is able to rapidly fabricate detailed patterns on protein based matrices with high fidelity as demonstrated in FIG. 10. To establish the efficacy of excimer laser ablation of collagen matrices, collagen gels at 2.5 mg/mL initial concentration, 4 mm initial thickness and layered 4 times, were constructed, and excimer ablated. Thermal denaturation was determined by analyzing thermal transitions of collagen, which showed no measurable denaturation, and analyzing collagen ultrastructure—noting the retention of bulk and ablation edge native D-periodicity, 67 nm banding patterns, and fibrillar structure. These results are similar to those reported in clinical practice with excimer laser ablation strategies yielding small (0.1-0.3 μm) regions of damage.

### Generation of Mechanically Compliant Protein Substrates Through Laser Ablation

Ablated substrates were embedded in recombinantly expressed elastin and mechanically tested. The chosen ablation scheme, a triangular waveform pattern with vertical strips resulted in highly compliant structures, wherein structures with approximating unity aspect ratios showed collagen strips extending and straightening prior to failure. It is possible that increased vertical strip thickness helps buttress collagen strips, keeping them anchored to the composite structure, aiding in in-plane extension. Conversely, thinner vertical strips could allow for twisting and out-of-plane bending of collagen waves which would consequently have a lower strain at failure, Design 2. As a consequence of large amounts of material removal, there was a decrease in ultimate tensile strength. However, tissue engineered microablated composites exhibited mechanical properties that mimic several tissues. The mechanical properties of unablated and microablated composites can be tuned comparing favorably to native tissue, e.g., cartilage, ligament, coronary artery, and carotid artery (1.76-2.64 MPa UTS). See Table 4.

TABLE 4
Mechanical properties of unablated and ablated collagen matrices

| | | | Young's |
|---|---|---|---|
| | Ultimate Tensile | Strain at Failure | Modulus |
| | Strength (MPa) | (%) | (MPa) |
| Unablated matrices | 13.3 ± 2.19 | 18.0 ± 3.73 | 93.7 ± 19.8 |
| Ablated matrices | 0.683-5.82 | 9.43-69.6 | 2.91-88.1 |
| Arteries | 1.4-11.1 | N.A. | 1.54 ± 0.33 |
| Veins | N.A. | N.A. | 3.11 ± 0.65 |
| Cartilage | 3.7-10.5 | N.A. | 0.7-15.3 |
| Ligament | 24-112 | N.A. | 65-541 |

Cell Supportive Matrices with Enhanced Global Alignment

Alignment of cells is important in recapitulation of native tissue structure and function. For example, Smooth muscle cells (SMCs) in the vascular media act to both maintain contractility during pulsatile blood flow, as well as vasoconstrict as a function of neuronal or chemokine action. Further, cells in the myocardium and several other muscle tissue align in the direction of physiologic stress to aid in bio-mechanical function and contractility.

An excimer-laser assisted ablation scheme creates ordered micro-ribbons that are intrinsically cell adhesive and potentially self-align cells. Through the use of microfabrication technology and excimer laser ablation, cell adhesive substrates were developed with precise micron-level patterns. This is a facile method for the alignment of confluent cell layers on collagen matrices produced in a matter of hours. Further, actin staining reveals that cellular cytoskeletal filament alignment is preferential in the direction of ablated waves, compared to unablated controls. Cellular alignment is uniform throughout matrices, allowing for rapid generation of large-scale cellularized tissue engineered substrates with structural, mechanical and cellular anisotropy. Compared to several cell alignment techniques in the field, microablation allows for the fabrication of thick constructs that are independent of potential nano-scale topographical inhomogeneities that may affect alignment on self-assembled monolayers or nano-/micro-patterned substrates. Another method for cellular alignment proposed is the spatial patterning of cell adhesive moieties on substrates to facilitate localization of cells; however given the high cost of such materials and potential for misfolding, lack of adequate moiety presentation, and surface inhomogeneities, such techniques are limited in scalability and translation to large scale tissue engineered products. Disclosed herein is a highly scalable and strong collagen that is cell adhesive resulting in alignment and providing significant mechanical strength similar to native tissue.

Matrices with Tunable Mechanical Properties and Tissue-Mimetic Microarchitecture

Mechanical failure of collagen gels has hampered efforts to fully utilize the properties of collagen. Through the use of fabrication techniques, enhanced dehydration and compaction of collagen gels, and co-gelling with recombinantly expressed elastin, a series of mechanically tunable matrices have been generated that support cell adhesion and proliferation while exhibiting mechanical properties that are similar to vascular and other soft tissue. Mechanical strength and stiffness can be varied through the judicious selection of initial gel formulations, concentrations, gel thicknesses, layering of matrices and crosslinking. The addition of another matrix component during gelation (elastin-mimetic polypeptides), which decorates collagen fibrils, causes an increase in strain to failure and increased strength of matrices. Enhanced mechanical compliance due to the ability of elastin-mimetic polypetides to disrupt the inter-fibrillar structure of collagen, may result in decreased collagen fibrillar branching and adhesions, allowing for enhanced fibril pullout and transition from brittle to ductile fracture. This further allows enhanced fibrillar slippage/pullout and withstanding additional force prior to formation of defects leading to failure.

Matrices of this type have been found in bone, that act as sacrificial bond forming matrices that allow for a “hidden length” of fibrils to be found, which may further explain the enhanced strain to failure of composite matrices over collagen only matrices. Additionally, elastinmimetic polypeptides may act as a “glue” to better adhere adjacent collagen fibrils, resulting in higher strengths. Hydroxyl groups and sulphydryl groups in collagen and recombinant elastin matrices enable micro-crosslinks, both physical and chemical (Van der Waal's, ester, thioester) to form between monomers during gel dehydration. Through the modulation of macroscale crosslinks, due to the addition of genipin, further strengthening of uncrosslinked matrices when crosslinked is noted. One may create mechanically resilient structures that exhibit high failure strain and enhanced compliance when used to form rolled tubes.

### Vascular Grafts

Cellularized production of dense collagen-elastin interpenetrating networks (IPNs) matrices is typically done over 24 h to ensure cell adhesion, proliferation and near confluence. Cells are allowed to proliferate on collagen matrices prior to embedding with elastin, rolling on a 1.3 mm or 4 mm mandrel and re-gelling the elastin to form one contiguous layer. This technique allows for the rapid generation of cellularized vascular grafts that have sufficient mechanical strength and stability for implantation. MSCs provide a convenient source for the population of vascular grafts, given their ease of isolation from a variety of sources (bone marrow, peripheral blood, adipose tissue). Mesenchymal stem cells have been shown to differentiate into endothelial progenitor like-cells and other vascular wall cellular constituents, including fibroblasts, and smooth muscle cells.

A small diameter vascular graft was developed for surgical implantation in a rat aortic interposition model. This vascular graft showed good success without note of occlusive thrombi or neointimal hyperplasia at one week. Integration and healing of the anastamoses was noted. CTA showed patency of grafts with no appreciable aneurysmal dilation. Histological evaluation showed no evident calcification, coverage of EC-like cells on the luminal surface, neo-collagen matrix synthesis, with minimal leukocyte infiltration and no occlusive thrombus presence.

### EXPERIMENTALS

### Generation of High Density Collagen Mats

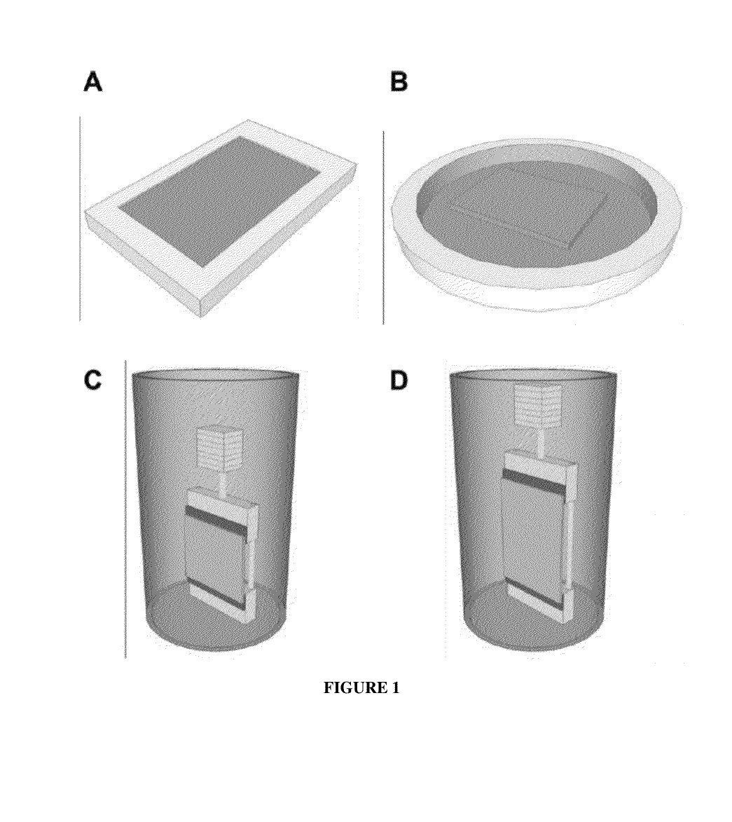

Centimeter scale collagen matrices can be mechanically tuned and anisotropically defined. Type I collagen was isolated from rat tail tendon and confirmed for purity by PAGE gel analysis. Collagen gels were structurally aligned, analyzed for native ultrastructure and tested for mechanical strength. Gels were synthesized at a variety of concentrations (0.3125 mg/mL, 0.625 mg/mL, 1.25 mg/mL and 2.5 mg/mL) by neutralization in a phosphate buffer for 24 hours at 4° C. Gel thickness was determined by the volume of total solution in 10 cm×8 cm rectangular molds. See FIG. 1A. Fibrillogenesis within collagen gels was enhanced by incubation in a fiber incubation buffer for 48 hours at 37° C. See FIG. 1B. For alignment of collagen matrices, gels were mounted on a axial stretcher and stretched to 0, 10, 20% strain at 3 or 300 μm/s. See FIG. 1C & D. Collagen gels were subsequently air dried to less than 1% of their initial thickness under a constant air stream, generating collagen mats. This technique may be used for the development of collagen matrices with structural anisotropy in a scalable method, generating non-denatured matrices suitable for tissue engineering.

### Fibrillar Microstructure and Preservation of Native Collagen Structure

Ultrastructural analysis of collagen mats showed uniformity of collagen fibril diameter for unaligned and aligned gels. 10 k×SEM images of critical point dried collagen matrices show uniformity and isotropy of unaligned matrices (FIG. 2 A). 50 k× magnification SEM images of critical point dried collagen matrices were used to measure collagen fibril diameter. See FIG. 2 B. Fibril diameter for unaligned 2.5 mg/mL gels was 83.1±9.44 nm, for 1.25 mg/mL gels was 75.7±14.8 nm and for 0.625 mg/mL gels was 74.3±11.4 nm. Fibril diameter for 20% aligned 2.5 mg/mL gels was 78.2±17.0 nm, for 10% aligned 2.5 mg/mL gels was 81.7±14.8 nm and for 20% aligned 0.3125 mg/mL gels was 88.52±11.7 nm, which showed no significant difference with alignment, stretch amount, stretch rate or concentration. TEM images of uranyl acetate stained collagen mats showed characteristic D-periodicity, 67 nm collagen banding patterns (FIGS. 2 C& D). Concentration variation did not significantly affect the fibril diameter or the ultrastructure of the collagen gels. Collagen mats which resemble native matrix in macro- and ultra-structure have been created through the neutralization of collagen gels using a phosphate based buffer. Incubation of the gels with fibril incubation buffer to promote fibrillogensis of collagen fibrils, and drying the gels into dense matrices.

Generation of Structural Anisotropy within Collagen Matrices

To enhance tissue mimetic architecture, it is required that matrices exhibit mechanical anisotropy to ensure matching of tissue based replacements. Subsequent to treatment in fiber incubation buffer gels were adhered onto plastic frames and mounted on an automated motorized stretching device (FIG. 1 C). Higher stretching rates (300 μm/s) resulted in an inability to generate structural anisotropy (FIG. 3 A). Lower stretching rates (3 μm/s) resulted in distinct fibril reorganization into defined structures (FIGS. 3B & C, Table 2.1). FFT analysis of 10 k×SEM images of collagen mats yield relative frequencies of fibrils from the horizontal axis. Fibril relative frequencies were summed in 5 degree increments and histograms were plotted as a function of angle. See FIGS. 3 D-F.

Histogram plots were then fitted with a Gaussian curve and FWHM was subsequently determined. FFT analysis of 10 k× magnification images of 300 μm/s strained samples to 10% or 20% showed no preferential alignment of collagen fibrils. See FIG. 3 D. Depending on the strain amount, 10% or 20%, the degree of alignment varied at a lower strain rate of 3 m/s, FIG. 3 E & F. Maximum alignment was achieved with 20% strain at a rate of 3 μm/s. Concentration variation did not significantly affect the amount of alignment, or the maximum alignment. Alignment for 2.5 mg/mL gels strained to 10% at a rate of 3 μm/s had a maximum of 5.64% with a FWHM of ±37.5°. Alignment for 2.5 mg/mL gels strained to 20% at a rate of 3 μm/s had a significantly higher maximum of 6.86% with a FWHM of ±35.2°. See FIG. 4. This data indicates the ability to modulate the alignment of collagen matrices as a function of strain rate and strain amount.

Mechanical Strength of Collagen Matrices with and without Alignment

In order to determine utility in a variety of soft tissue engineering applications, the strength of collagen matrices were determined as a function of alignment. Collagen gels of various concentrations from 0.3125 mg/mL-2.5 mg/mL were aligned, dried and crosslinked with genepin. Uniaxial stress-strain testing collagen mats were performed using a DMTA V mechanical tester. Rectangular strips, 20 mm×5 mm were cut from sheets of unaligned and aligned matrices in the direction of alignment, and perpendicular to alignment. Sheets were mounted vertically on the testing platform and immersed in PBS at 37° C. Samples were preconditioned and tested to failure. Samples that failed at the mounting points and those that slipped were discounted from analyses. Mechanical anisotropy was noted correlating to structural anisotropy. The mechanical strength, ultimate tensile strength (UTS), and stiffness, Young's modulus (Mod.), of aligned collagen matrices were significantly higher than that of unaligned samples, irrespective of concentration. Aligned matrices showed an approximate doubling in mechanical strength independent of concentration, for 2.5 mg/mL gels from ˜3.50 MPa to 8.00 MPa. See Table 1.

TABLE 1
Consolidated mechanical and structural properties of collagen mats cast at
different initial concentrations and aligned to different amounts.

| | | | | | | Maximum | | |
|---|---|---|---|---|---|---|---|---|
| Initial gel | Fibril | | | Strain at | | relative | | |
| conc. | diameter | Mat | UTS | failure | Young's | frequency | | % Stretch |
| (mg/mL) | (nm) | thickness | (MPa) | (%) | modulus | of fibrils | FWHM | Alignment |
| 0.3125 | 88.4 ± 12.5 | 26.4 ± 1.42 | 3.71 ± 0.716 | 10.0 ± 1.19 | 43.1 ± 7.80 | NA | NA | 0 |
| 0.625 | 74.3 ± 11.4 | 27.6 ± 1.04 | 3.25 ± 0.31 | 10.5 ± 1.25 | 44.7 ± 8.21 | NA | NA | 0 |
| 1.25 | 75.7 ± 14.8 | 28.5 ± 2.68 | 3.27 ± 0.400 | 11.2 ± 1.60 | 42.7 ± 3.51 | NA | NA | 0 |
| 2.5 | 83.1 ± 9.44 | 26.7 ± 2.58 | 3.50 ± 0.478 | 10.4 ± 1.73 | 38.7 ± 9.37 | NA | NA | 0 |
| 0.3125 | 88.5 ± 11.7 | 24.9 ± 2.03 | 7.57 ± 0.682 | 11.2 ± 1.04 | 98.3 ± 15.2 | 6.65 ± 0.237 | 35.4 ± 2.26 | 20 |
| 0.625 | 85.4 ± 12.2 | 25.7 ± 2.06 | 7.43 ± 0.564 | 10.2 ± 1.24 | 91.4 ± 10.7 | 6.69 ± 0.206 | 35.1 ± 1.31 | 20 |
| 1.25 | 83.4 ± 9.26 | 26.3 ± 1.86 | 7.49 ± 0.639 | 10.7 ± 1.55 | 92.7 ± 6.23 | 6.63 ± 0.180 | 36.3 ± 1.36 | 20 |
| 2.5 | 81.7 ± 14.8 | 28.0 ± 1.28 | 6.20 ± 1.04 | 10.8 ± 1.83 | 79.8 ± 15.7 | 5.56 ± 0.213 | 37.5 ± 3.25 | 10 |
| 2.5 | 78.2 ± 17.0 | 25.2 ± 1.03 | 8.00 ± 1.17 | 9.85 ± 1.46 | 103 ± 15.6 | 6.75 ± 0.209 | 35.5 ± 2.10 | 20 |

Further, stiffening of matrices occurred, resulting in Young's Moduli increases for 2.5 mg/mL gels from 38.7 MPa to 103 MPa. See Table 1. Mechanical strength was a function of alignment with 10% aligned matrices having significantly lower UTS and Young's Moduli than 20% aligned matrices, FIGS. 5 A & C, Table 1. The mechanical strength in the direction perpendicular to that of alignment in anisotropic samples was not significantly different than unaligned samples. See FIG. 5. Increase in stretch amount of 2.5 mg/mL matrices from 10% to 20% resulted in a greater amount of alignment, and consequently in significantly higher strength and stiffness (p<0.05). Further, this correlated with maximum relative frequency of fibrils, which was significantly different for unaligned, 10% aligned and 20% aligned matrices, although the distribution of fibrils from the peak, FWHM, was not. Strain to failure of collagen matrices did not change significantly as a function of alignment, FIG. 5 B. Collagen matrices did not show a significant difference in fibril diameter as concentration or alignment varied, ranging from 74.3±11.4 nm to 88.5±11.7 nm. Further, concentration of initial gels did not significantly affect dried mat thickness. See FIGS. 5 D & E and Table 1. This data indicates the ability to modulate mechanical strength as a function of strain amount and strain rate. Further, microstructural alignment of collagen fibrils provides structural and mechanical buttressing during mechanical testing.

### Isolation and Purification of Monomeric Collagen

Acid-soluble, monomeric rat-tail tendon collagen (MRTC) was obtained from Sprague-Dawley rat tails. Frozen rat tails (Pel-Freez Biologicals, Rogers, Ak.) were thawed at room temperature and tendon was extracted with a wire stripper, immersed in 10 mm HCl (pH 2.0; 150 mL per tail) and stirred for 4 h at room temperature. Soluble collagen was separated by centrifugation at 30,000 g and 4° C. for 30 min followed by sequential filtration through P8, 0.45 μm, and 0.2 μm membranes. Addition of concentrated NaCl in 10 mm HCl to a net salt concentration of 0.7 m, followed by 1 h stirring and 1 h centrifugation at 30,000 g and 4° C., precipitated the collagen. After overnight re-dissolution in 10 mm HCl the material was dialyzed against 20 mm phosphate buffer for at least 8 h at room temperature. Subsequent dialysis was performed against 20 mm phosphate buffer at 4° C. for at least 8 h and against 10 mm HCl at 4° C. overnight. The resulting MRTC solution was stored at 4° C. for the short-term or frozen and lyophilized.

### Collagen Mat Fabrication Process

An acidic collagen solution (5 mg/mL in 10 mm HCl) is neutralized in a phosphate buffer (WSB: 10 wt % poly(ethylene glycol) Mw=35,000, 4.14 mg/mL monobasic sodium phosphate, 12.1 mg/mL dibasic sodium phosphate, 6.86 mg/mL TES (N-tris(hydroxymethyl) methyl-2-aminoethane sulfonic acid sodium salt), 7.89 mg/mL sodium chloride, pH 8.0), to yield large (centimeter scale) gels. Typically this is performed in rectangular molds to create rectangular gels of 5-10 cm on a side and 4 mm of thickness, but a wide range of dimensions are feasible. The gels are next subject to a 48 hr incubation in a fibril incubation buffer (FIB: 7.89 mg/mL sodium chloride, 4.26 mg/mL dibasic sodium phosphate, 10 mm Tris, pH=7.4).

The gels are allowed to dry in air, creating a mat of dramatically reduced thickness and elevated density. For example, a 4 mm hydrated gel is about 10 microns thick after drying into a mat. The length and width of the gel does not change as it is dried into a mat. At the nanoscale these mats are comprised of networks of collagen nanofibers (d=80 nm). Crosslinking agents, e.g., glutaraldehyde, genipin, or other conditions, e.g., dehydrothermal conditions or ultraviolet light exposure, can optionally be applied to the mats to increase strength and stability. Discussed below are modifications to the process that enhance the mechanical properties and utility of the mats.

### Stretch Alignment/Generation of Structural Anisotropy

Prior to drying, gels were mounted on an automated motor-driven expandable rack, submerged in a buffer solution. The rack stretched the gels along a single axis to various strains and at controlled rates of strain. Collagen gels (100×80×4 mm) were adhered onto 125 μm thick plastic frames, and mounted onto a motorized vertical stretching device (FIG. 1). The stretcher was expanded uniaxially at 3 μm/s and 300 μm/s to strains of 0, 10, 20% in deionized water, 25° C. Strains larger than 20% resulted in tearing of the collagen gels. Aligned gels were then air dried under constant tension for 24 h at 25° C., resulting in a dense collagen mat.

After drying, scanning electron microscopy indicated that the stretching process caused the nanofibers to align in the direction of stretch. Furthermore, mechanical testing showed that the mats were stronger and stiffer in the direction of stretch (parallel to fiber alignment) and weaker and less stiff in the perpendicular direction (the cross-fiber direction). Engineering strain levels of 10% and 20% were found to be effective at aligning the nanofiber structure, with more strain having a greater effect. A strain rate of 3 μm/sec was found to be effective, while a faster rate of 300 μm/sec was not effective for aligning the nanofibers.

The stretch alignment modification is useful because it:

(i) Increases the strength of the collagen mats (in one direction).

(ii) Results in anisotropic mechanical properties. Many native tissues exhibit mechanical anisotropy, so this material may be a closer replacement for those tissues.

(iii) Potentially will influence cell behavior.

### Crosslinking and Mechanical Testing of Constructs

Anisotropic and isotropic collagen mats were crosslinked in a biocompatible crosslinker, genipin-PBS (Fisher Scientific) at 6 mg/mL for 24 hours at 37° C. Samples were then cut into 20 mm long×5 mm wide rectangles that were mounted onto a Dynamic Mechanical Thermal Analyzer V (DMTAV, Rheometric Scientific, Piscataway, N.J.) with a gauge length of 10 mm, immersed in PBS at 37° C. and preconditioned 15 times to 66% of the average maximum failure strain for the sample, and then tested to failure at 5 mm/min. A total of 8 samples were tested for each group. Thickness of hydrated sample was measured using optical microscopy and then correlated to mechanical data to determine the ultimate tensile strength and strain at failure. Young's modulus was determined from the slope of the last 4% of the stress-strain curve.

### Layering of Collagen Gels

In this process, a hydrated collagen gel is placed upon a second, dried mat and allowed to dry, or a dried mat may be rehydrated and dried upon another dried mat. Following drying, the mats are firmly adhered and do not separate even upon re-hydration. Several layers (2, 4, 8 or more) may be stacked and dried in this way. The resulting multi-layer mats exhibit an unexpected increase in mechanical strength. For example, when two mats are dried together, it would be anticipated that the strength (force at failure when pulled in tension) of the double mat would be twice that of the single mat. However, data herein shows that in this scenario the force at failure increases by a factor of four or more. This modification is useful because of the increased strength. Also, this modification is useful because it allows for the creation of tubes from rectangular mats. To create a tube, a hydrated mat is rolled several times (2 to 6 or more) around a cylindrical support, or mandrel, and allowed to dry. After drying, a tube whose walls consist of multiple layers is generated. These tubes are expected to be useful for replacing tubular tissues such as blood vessels or other conduit structures.

Most processes in the literature rely upon collagen gels, which are weaker than the dense, dried collagen mats described here. In particular, the notable increase in strength observed after stacking the mats and drying them together was unexpected.

### Mechanical and Laser Patterning

Laser cutting of microscopic patterns and mechanical hole punching was used to create a variety of patterns in the mats that alter mechanical properties. Laser cutting of wavy patterns increased compliance and a led to a larger strain at failure. Data also indicates that microablation of round holes (diameter approximately 50 μm) resulted in increased suture retention strength (the force required to pull a suture out of the material). Mats have been patterned with excimer and CO₂ laser cutting equipment. Data indicates that no noticeable denaturation (destruction of the macromolecular structure of the collagen molecule) occurred when ablated with an excimer laser.

This modification is useful because:

(i) It can increase the compliance of the mats, making the material more stretchy and flexible. For the replacement of some soft tissues, especially blood vessels, it is desirable to match the high compliance of natural tissues.

(ii) Certain patterns may also permit the fabrication of kink-resistant blood vessel replacements (i.e. tubes that can be bent to a high level of curvature while not kinking).

(iii) Certain patterns may increase the suture retention strength of the material.

(iv) Certain patterns may also result in the alignment of cells that adhere and grow on the material.

(v) Certain patterns may improve the pattern of host tissue infiltration (for example by allowing host cells, capillaries, and secreted networks of matrix protein to infiltrate more quickly into the ablated areas).

### Excimer Ablation of Collagen Mats

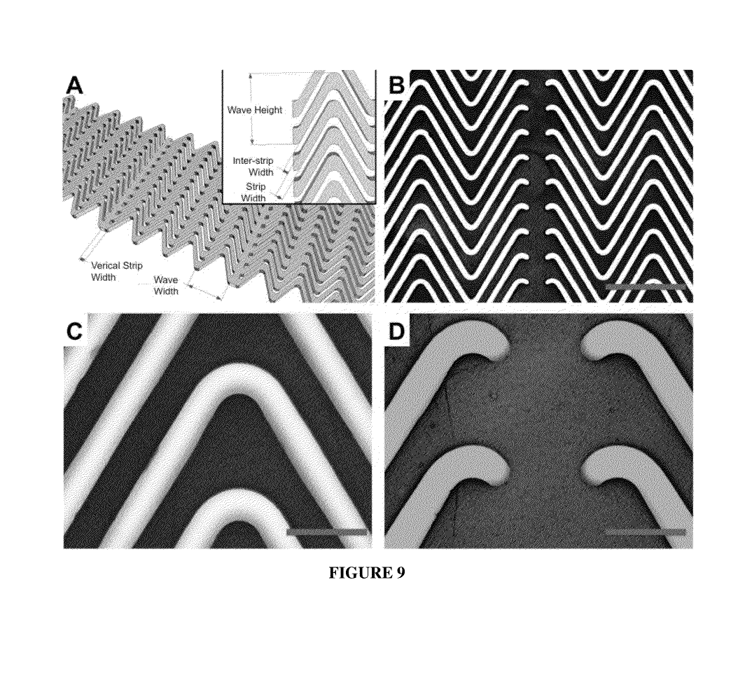

Different mask types were used, e.g., stainless steel masks and quartz masks. Stainless steel masks were constructed by infrared laser ablation of 50 μm thick stainless steel sheet stock. Quartz contact masks (Advance Reproductions, MA) were fabricated using photolithography and wet etching of 5 μm thick aluminum coated quartz. Five designs consisting of linear or sawtooth ablation patterns were investigated with geometric design variables consisting of strip length, strip width, interstrip gap and vertical strip width. These variables ultimately dictated the frequency and amplitude of the resultant waveform. See FIG. 9. The mask was placed over collagen matrices and ablated with an excimer laser with parameters adjusted to yield a fluence of 26.7 J/cm2 (Microelectronics Research Center at Georgia Tech, Atlanta, Ga.).

### Microdifferential Scanning Calorimetry of Collagen Mats

To determine the effect of mat fabrication and excimer laser ablation on collagen triple helical structure, thermal denaturation temperature and enthalpy of denaturation were measured using a differential scanning calorimeter (μDSC, SETARAM, Pleasanton, Calif.). Briefly, 5-10 mg segments of lyophilized collagen, dried collagen mats pre or post excimer ablation, and post crosslinking in genipin were hydrated in 0.5 mL of PBS for 10 h at 5° C. Mats were then heated from 5° C. to 90° C. and back to 5° C. at 0.5° C./min. The enthalpy of phase changes relating to denaturation, HD, was measured, as well as the denaturation temperature, TD. Complete denaturation was confirmed by the lack of a denaturation peak upon a repeated heating (to 90° C.) and cooling cycle.

Combining Collagen Mats with ELP.

The flanking 75 kDa endblocks of the protein polymer contained 33 repeats of the hydrophobic pentapeptide sequence [IPAVG]₅, and the central 58 kDa midblock consisted of 28 repeats of the elastic, hydrophilic sequence [(VPGAG)₂VPGEG(VPGAG)₂]. Additional sequences between blocks and at the C terminus include the residues [KAAK], which along with the N-terminal amine provide amino groups for chemical crosslinking.

The protein polymer sequence is contained in a single contiguous reading frame within the plasmid pET24-a, which was used to transform the Escherichia coli expression strain BL21(DE3). Fermentation was performed at 37° C. in Circle Grow (QBIOgene) medium supplemented with kanamycin (50 μg/mL) in a 100 L fermentor at the Bioexpression and Fermentation Facility of the University of Georgia-Athens. Cultures were incubated under antibiotic selection for 24 h at 37° C. Isolation of the LysB10 consisted of breaking the cells with freeze/thaw cycles and sonication, a high speed centrifugation (20,000 RCF, 40 min, 4° C.) with 0.5% poly(ethyleneimine) to precipitate nucleic acids, and a series of alternating warm/cold centrifugations. Each cold centrifugation (20,000 RCF, 40 min, 4° C.) was followed by the addition of NaCl to 2 m to precipitate the protein polymer as it incubated for 25 min at 25° C. This was followed by warm centrifugation (9500 RCF, 15 min, 25) and resuspension of the pellet in cold, sterile PBS on ice for 10 to 20 min. After 6 to 10 cycles, when minimal contamination was recovered in the final cold centrifugation, the material was subject to a warm centrifugation, resuspended in cold sterile PBS, dialyzed, and lyophilized. Lyophilized protein was resuspended in sterile molecular grade water at 1 mg/mL and endotoxin levels were assessed according to manufacturer instructions using the Limulus Amoebocyte Lysate (LAL) assay (Cambrex). Levels of 0.1 EU/mg were obtained (1 EU=100 pg of endotoxin), which corresponds to endotoxin levels for clinically used alginate (Pronova sodium alginate, endotoxin ≦100 EU/g)

When the initial acidic collagen solution is prepared, other compounds can be included, including ELP. ELP are highly soluble in cold aqueous solutions, so can be added in a wide range of concentrations (up to ˜150 mg/mL). However, ELP comes out of solution at about 15° C. and forms a gel. Therefore, after a cool (about 4° C.) acidic collagen-ELP solution is neutralized to gel the collagen component, the gel may be warmed to cause the ELP component to also gel. This composite gel, or interpenetrating network (IPN), can then be dried and further processed similarly to a collagen mat.

In a second approach to combining collagen and ELP, the ELP can be used to glue multiple layers of collagen mat together. For example, a cool ELP solution can be placed in between stacked collagen mat layers, and allowed to permeate into the mats. When the stack is warmed, the ELP solution transitions into a gel, both within and between the mats, and the mat layers are adhered together. This gluing process can similarly be used to roll a mat layer about a central supporting mandrel multiple times and glue it to form a tube.

This modification is useful because:

(i) It presents another way to laminate mats and create tubes.

(ii) It may increase the tensile strength, suture retention strength, and compliance of the mats.

(iii) It may improve the blood contacting behavior of the mats. ELP surface coatings have been shown to have desirable blood-contacting behavior (little clotting results when blood contacts an ELP-coated surface). In contrast, collagen causes blood to clot. Therefore, an IPN or other ELP coating may reduce or eliminate the clotting.

### Generation of Collagen Mats and Nanofibrous Composites

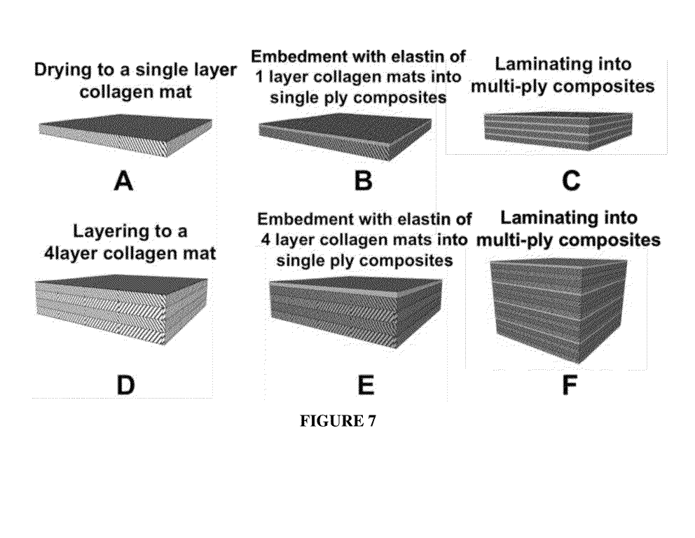

Collagen materials with high strength and tunable mechanical properties were generated in single and multiple layers. See FIG. 7. Multi-layer mats, generated by the serial drying process, were well integrated with no distinguishable interface between layers. Mechanical peeling of mats resulted in whole tears, without the ability to tweeze out individual layers of multi-layer mats. Collagen mats, due to their fibrillar nature, allow for impregnation with alternative matrices that can modulate mechanical or biological behavior. The sandwich molding technique permitted the infusion of ELP into collagen matrices, leading to nanofibrous composite matrices, schematically shown in FIGS. 7 B, C, E, & F. Dry matrices before and after the addition of ELP had spatial densities of 0.772±0.0626 mg/cm² or 0.983±0.0558 mg/cm², respectively, suggesting the composite matrices are 78.5% collagen and 21.5% ELP by dry weight. In addition to the single- and multi-layer mats described above, the ELP molding process allowed the formation of single- and multi-ply structures. See FIG. 7.

### Mechanically Tunable Collagen Mats as a Function of Concentration, Thickness and Layering

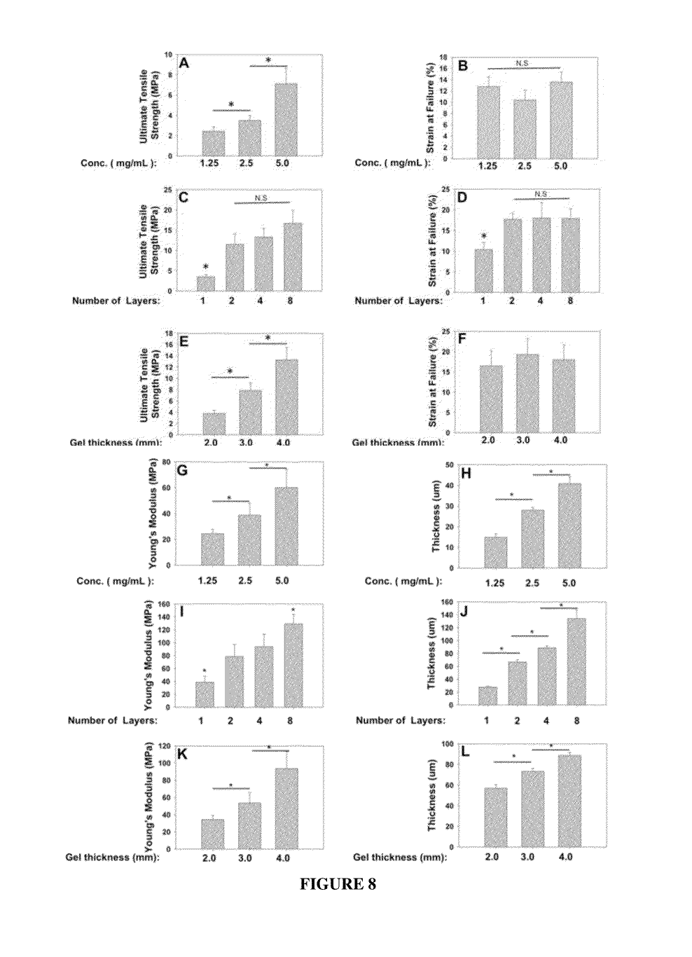

Initial collagen constructs showed a significant increase in strength and stiffness of matrices as a function of concentration, but not a significant difference in strain at failure, FIGS. 8 A, B & G. When compared to single layer matrices, multilayer matrices showed an increase in strength (4-14 MPa), strain at failure (10-17%) and stiffness (40-100 MPa), FIG. 8 C, D & I. Increasing gel thickness from 2 to 4 mm prior to drying resulted in collagen mats of increasing strength and stiffness, with no significant effect on strain-at-failure. See FIGS. 8 E, F & K. Individual collagen mats had a nominal thickness of 14.9-40.8 μm depending on initial collagen concentration in the gels. See FIG. 8 H. Layering of collagen gels showed a commensurate near-linear increase of thickness. See FIG. 8 J.

### Development of Structurally and Mechanically Anisotropic Collagen Microarchitectures

Excimer laser ablation permits the use of a variety of masking techniques to ablate almost any design onto collagen substrates. Critical features of the triangular waves designs, described herein, included wave height, strip width, inter-strip width, wave width, and vertical strip width, FIG. 9 A and Table 2.

TABLE 2
Thermal properties of collagen matrices

| | | | | Excimer- | Genipin |
|---|---|---|---|---|---|
| | Monomeric | Collagen | | treated | crosslinked |
| | collagen | lyophilized | Collagen mat | collagen mat | collagen mat |
| T_{D} (° C.) | 36.2 ± 0.6 | 46.0 ± 0.521 | 52.9 ± 0.396 | 53.1 ± 0.203 | 73.2 ± 2.11 |
| ΔH (J/g) | 49.4 ± 0.8 | 47.8 ± 4.77 | 44.0 ± 3.21 | 48.2 ± 1.32 | 27.3 ± 1.89 |

The fidelity and resolution of the excimer laser allows for exact cuts to be made into the collagen mats. See FIG. 9 B, C, D. Although the theoretical resolution of the excimer laser is 248 nm, the practical resolution is typically higher. Consequently minimum feature sizes were 10 μm.

### Mechanical Testing of Composites

To simulate application in planar soft tissues, collagen sheets (with and without microablation) were cut into 20 mm×5 mm strips and mounted onto a Dynamic Mechanical Thermal Analyzer V (DMTA V, Rheometric Scientific, Piscataway, N.J.) with a gauge length of 10 mm, immersed in PBS at 37° C. Samples were preconditioned 15 times to 66% of the average maximum failure strain determined from pilot samples, and then tested to failure at 5 mm/min (n=8 for each group). Hydrated thickness was measured using optical microscopy for calculation of cross-sectional area. Young's modulus was determined from the slope of the last 4% of the stress-strain curve. Suture retention strength of planar constructs was determined by cutting 4 mm×4 mm square inserting 4-0 FS-2 prolene suture (Ethicon) through the center of the segment, and pulling out the suture with force measured on the DMTA (n=4 for each design).

### Preservation of Collagen Macromolecular Structure

Thermal analysis showed that the denaturation temperature of lyophilized collagen was lower than uncrosslinked and crosslinked collagen mats, 46.0±0.5° C., 52.9±0.4° C. and 73.2±2.1° C., respectively. See Table 2. Lyophilized collagen consisted of monomeric collagen prior to higher order assembly, thus exhibiting a lower TD than collagen mats, which were treated with phosphate buffer. Further the ion concentrations, pH and heating rate (in addition to buffer type) contribute to collagen monomer organization into larger fibrils. Additionally, changes in ultrastructure conferred during phosphate buffer treatment and densification of the matrix during mat fabrication contribute to a higher TD for collagen mats. Lyophilized collagen and collagen mats exhibited similar HD. Crosslinking of matrices results in a greater stabilization of the collagen structure and consequently raises the TD, but lowers HD. There was no significant difference in the thermal transitions or enthalpy between collagen mats with and without ablation suggesting no measurable loss in triple helical structure.

### Design of Mechanically Variant Structures for Optimized Mechanical Compliance

Ablation techniques involved ablation of holes 10-100 μm in diameter, direct write of lines and waves, and variations of the designs listed in Table 3.

TABLE 3
Design variations for ablated collagen mats.

| | | | | | | Vertical |
|---|---|---|---|---|---|---|
| | | | | Angle of | Strip aspect | Strip |
| | Wave strip | Wave strip | Interstrip | wave crest | ratio | thickness |
| Design | length (μm) | width (μm) | width (μm) | (°) | (Height:Width) | (μm) |
| 1 | 2000 | 120 | 10 | 0 | 0.5 | 100 |
| 2 | 500 | 60 | 30 | 60 | 1 | 60 |
| 3 | 500 | 60 | 10 | 60 | 1 | 100 |
| 4 | 500 | 60 | 10 | 60 | 1 | 300 |
| 5 | 500 | 60 | 10 | 60 | 1 | 600 |

It was discovered that the strip width to height (film thickness) ratio needs to be approximately <1 to ensure features are stable and do not laterally collapse during subsequent processing. Further, it was determined that thick wave strips (>180 μm) resulted in out of plane bending of wave features. Consequently, the subset of designs that resulted in improvements of mechanical properties is shown in Table 2. Linear ablation patterns were also generated to determine the altered mechanical response as a function of excimer laser ablation pattern. Wave patterns with varied vertical strip thickness 60-600 μm and interstrip thickness with variation from 10-30 μm demonstrate the modulation of mechanical strength and suture retention strength.

Collagen Mat Ablation Closely Mimics Mask Features with No Protein Denaturation

Metal masks (stainless steel shim stock, 50 μm), FIG. 10 B, or aluminum coated quartz, FIGS. 10 A, C-E, allow laser transmission through 10-30 μm gaps, showing high ablation fidelity, allowing patterns on the centimeter scale to be completely ablated over a period of less than 1 h. Collagen wave ablation shows high precision and uniformity under SEM, FIG. 10 K, which is composed of a nanofibrous (80 μm) fibrillar matrix, FIG. 10 L & M. To demonstrate regeneration and reconstitution of native collagen structure, in addition to the differential scanning calorimetry described above, native collagen banding structure is noted in the matrix bulk. See FIG. 10 N, and edge of waves, FIG. 10 O.

### Mechanical Properties of Ablated Composites

The utility of excimer laser ablation to modulate stiffness and extensibility is shown in FIG. 11. Linear ablation patterns result in a slightly greater than 50% reduction in tensile strength from unablated matrices, 5.82±0.93 MPa vs 13.3±2.19 MPa. See FIG. 8 C vs FIG. 11A. In the triangular wave designs, increasing vertical strip width enhanced ultimate tensile strength. With other features constant, vertical strip width ranging from 100 μm, 300 μm and 600 μm (designs 3-5), had UTS of 0.958±0.172 MPa, 1.20±0.296 MPa, 1.43±0.162 MPa, respectively. This trend is maintained with collagen waves in Design 2 which had a significantly lower UTS of 0.683±0.168 MPa, a thinner vertical strip thickness, 60 μm, and waves spaced further apart, 30 μm. Triangular patterning tended to increase strain at failure, from 9.43±1.76% for linear ablation patterns (Design 1) to 44.5±8.27%, 51.8±14.4%, 65.9±8.19%, and 69.6±10.9% for Designs 2-5, respectively. Further, there is a significant increase in the strain at failure for Designs 4 and 5 over Design 2. The Young's modulus of linear ablated constructs is significantly higher than that of triangular wave patterned collagen, 88.1±12.9 MPa, compared to 3.95±0.839 MPa, 2.92±0.579 MPa, 5.24±1.00 MPa, 5.06±1.34 MPa for Designs 2-5, respectively. Suture retention strengths for 4 layer composites, stacked into 4 ply systems with ELP, showed suture retention strength of 52.4±9.18 gF. However, ablated constructs, which have less collagen, had suture retention strengths of 51.2±7.43 gF, 37.7±12.1 gF, 40.1±5.88 gF, 36.36±6.23 gF and 37.3±5.48 gF for Designs 1-5, respectively.

### Imaging of Composite Architecture

Optical microscopy, fluorescence microscopy, scanning electron microscopy (SEM), and transmission electron microscopy (TEM) were used to analyze the collagen structure pre and post embedment in elastin. For SEM studies, briefly, dry collagen mats were hydrated in water for 24 h and dehydrated in serial exchanges of ethanol-water mixtures from 30%-100%. The samples were then critical point dried (Auto Samdri 815 Series A, Tousimis, Rockville, Md.), sputter coated with 8 nm of gold (208HR Cressington, Watford, England) and imaged at an accelerating voltage of 10 keV using a field emission scanning electron microscope (Zeiss Supra FE-SEM, Peabody, Mass.). To determine the ultrastructure and presence of D-periodicity in the fibrils, showing maintenance of native collagen structure, hydrated samples were prepared for TEM. Samples in PBS were washed in 0.1M cacodylate buffer and fixed in glutaraldehyde. After washing in water, samples were partially dehydrated in ethanol and stained with uranyl acetate. Samples were then fully dehydrated in ethanol, embedded in resin and polymerized. Ultrathin (60-80 nm) were cut using a RMC MT-7000 ultramicrotome (Boeckeler, Tucson, Ariz.). Post-staining with uranyl acetate and lead citrate was followed by imaging using a JOEL JEM-1400 TEM (JOEL, Tokyo, Japan) at 90 kV.

Combining Collagen Mats with Living Cells

Collagen mats are seeded with living cells. Specifically, stacked sheets and rolled tubes are created with bone marrow mesenchymal stem cells, but a wide range of cell types are likely to survive and proliferate on the mats. This modification is useful for creating biomaterials with the potential to grow and remodel, or demonstrate other types of bioactivity limiting the host's inflammatory response, reducing the spread of infection, or otherwise improving biocompatibility following implant. Anti-inflammatory and antibacterial drugs may be added to the mats. Adding minerialized hydroxyapetite and calcium phosphates may be used to create a bone substitute or for creation of hard tissue substitutes.

Rat Mesenchymal Stem Cell (rMSC) Cell Culture

Bone marrow-derived rMSCs (Stice lab, University of Georgia, GA) were seeded onto collagen constructs to establish cytocompatibility. Collagen scaffolds with and without microablation were sterilized in 70% ethanol for 30 min, washed several times in 1×PBS, and incubated in media for 30 min prior to cell seeding. Cells were cultured in Alpha MEM, supplemented with 10% fetal bovine serum, 1% L-glutamine and 1% penicillin-streptomycin. Cells were removed from tissue culture-treated polystyrene flasks using 0.25% trypsin-EDTA, suspended in media, and seeded at a concentrations of 100 000 cells/cm2 for 24 h. Assessment of cellular viability and alignment. Cell adhesion and morphology was probed using Live/Dead staining (Invitrogen, Carlsbad, Calif.), and Alexa Fluor® 568 phalloidin (Invitrogen, Carlsbad, Calif.), as per manufacturer's protocol. For Live/Dead staining, scaffolds were washed 3 times in PBS without divalent salts, and incubated with 2 mL of Live/Dead stain (2 M calcein AM and 4iM Ethidium homodimer-1 solution in PBS) for 1 hour. Scaffolds were then placed on glass slides with the addition of 20 L of Live/Dead stain and coverslipped. Stained cells were imaged using a Lecia SP5 confocal coupled with a white light laser and adjustable emission collectors (Leica, Buffalo Grove, Ill.). Calcein AM was imaged using excitation of 488 nm and emission of 518 nm, and Ethidium homodimer-1 was imaged at an excitation of 528 nm and emission of 617 nm. For cellular alignment, actin filament organization was probed. Briefly, scaffolds were washed with PBS, fixed in 4% buffered paraformaldehyde, washed in 0.5% Triton X in PBS, washed in 100 mM glycine in PBS, blocked with 1% BSA in PBS, and stained with Alexa Fluor 568 phalloidin dissolved in methanol. Excess stain was washed in PBS. Scaffolds were mounted onto glass slides, 20 μL of DAPI Prolong Gold® (Invitrogen, Carlsbad, Calif.) was added and coverslipped. Scaffolds were imaged after 24 h using a Leica SP5XMP inverted confocal microscope (Leica, Buffalo Grove, Ill.) coupled with a white light laser and 405 nm diode laser. DAPI was imaged using excitation of 405 nm and emission of 461 nm, and phalloidin was imaged using excitation of 578 nm and emission of 600 nm.

### Structural Features Dictate Cellular Alignment

Adhesion and spreading of rMSCs on microablated collagen matrices was observed within 4 h and proliferation in 24 h, FIG. 13 A, at low seeding densities, 100,000 cells/cm2. This provides a method to enhance global alignment of cells on microablated matrices, as seen in Live/Dead staining and staining of cytoskeletal actin filaments, FIG. 13 B & C.

### Production of Dense Collagen-Elastin Interpenetrating Networks (IPNs)

Monomeric rat tail tendon collagen and Lys-B10 were dissolved in 10 mM HCl, at concentrations ranging between 0.6125 mg/ml-5.0 mg/ml and various collagen and elastin ratios. Mixtures were neutralized using a gelation buffer (4.14 mg/ml monobasic sodium phosphate, 12.1 mg/ml dibasic sodium phosphate, 6.86 mg/ml TES (N-tris(hydroxymethyl) methyl-2-aminoethane sulfonic acid sodium salt, 7.89 mg/ml sodium chloride, pH 8.0) at 4° C. and were poured immediately into rectangular molds (10×8×0.4 cm) for 24 h. Gels were subsequently placed in a fiber incubation buffer (7.89 mg/ml sodium chloride, 4.26 mg/ml dibasic sodium phosphate, 10 mM Tris, pH 7.4) at 37° C. for 48 h to promote collagen fibrillogenesis. Gels were then dried at room temperature under a steady air stream. Stacked IPN mats consisting of 2 to 4 layers were generated by serially drying additional gels on top of dried mats. Some specimens were crosslinked in genipin in 1×PBS at 37° C. for 24 h.

### Imaging of Composite Architecture

Optical microscopy, fluorescence microscopy, scanning electron microscopy (SEM), and transmission electron microscopy (TEM) were used to analyze the collagen structure pre and post embedment in elastin. For SEM studies, briefly, dry collagen mats were hydrated in water for 24 h and dehydrated in serial exchanges of ethanol-water mixtures from 30%-100%. The samples were then critical point dried (Auto Samdri 815 Series A, Tousimis, Rockville, Md.), sputter coated with 8 nm of gold (208HR Cressington, Watford, England) and imaged at an accelerating voltage of 10 keV using a field emission scanning electron microscope (Zeiss Supra FE-SEM, Peabody, Mass.). To determine the ultrastructure and presence of D-periodicity in the fibrils, showing maintenance of native collagen structure, hydrated samples were prepared for TEM. Samples in PBS were washed in 0.1 M cacodylate buffer and fixed in glutaraldehyde. After washing in water, samples were partially dehydrated in ethanol and stained with uranyl acetate. Samples were then fully dehydrated in ethanol, embedded in resin and polymerized. Ultrathin (60-80 nm) samples were cut using a RMC MT-7000 ultramicrotome (Boeckeler, Tucson, Ariz.). Post-staining with uranyl acetate and lead citrate was followed by imaging using a JOEL JEM-1400 TEM (JOEL, Tokyo, Japan) at 90 kV.

Cellularization of IPN/Composite Sheets with Rat Bone Marrow Derived Mesenchymal Stem Cells (rMSCs).

IPN mats were sterilized in 70% ethanol for 30 mins. Scaffolds were dried and washed multiple times in PBS and incubated in media prior to seeding with cells. rMSCs were cultured in T75 flasks (Corning LifeSciences, Corning, N.Y.) for 3-5 days until near confluence. Cells were used between the 3rd and 5th passage. Cells were trypsinized and resuspended at concentrations of 50,000, 100,000, and 200,000 cells/cm² in full media. Cells were seeded on collagen constructs for 4 h, 12 h and 24 h. Live/Dead™ staining (Invitrogen, Carlsbad, Calif.) and subsequent confocal microscopy (Leica SP5XMP) was performed on constructs, to determine optimal seeding time for confluence of cells on scaffolds. A subset of small diameter grafts (0.9 mm ID) were seeded with MSCs and murine dermal microvascular endothelial cells by infusion in the lumen or seeding of adventitia with cells. For cell coverage quantification, 10× magnification images at 2048×2048 pixel resolution were obtained. A MATLAB script was written that decomposed red, green and blue layers from the images. Green images (live cells) were then thresholded based on script input and spatial coverage of cells per field determined.

### Fabrication of Acellular and Cellularized Collagen-Elastin Nanofibrous Grafts.

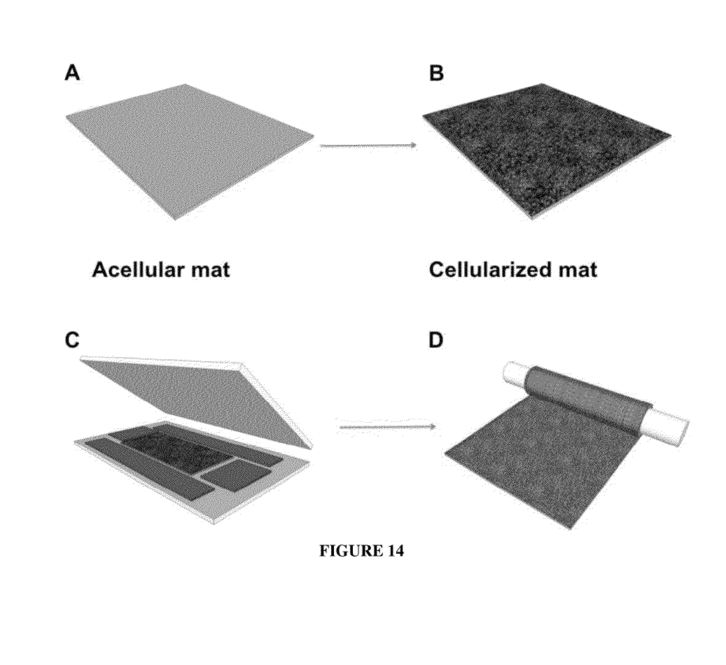

The overall schematic for the design, cellularization, and construction of the vascular grafts is outlined in FIG. 14. A solution of collagen and recombinantly expressed elastin were gelled, seeded with cells as desired, embedded in recombinant elastin, and rolled into tubes. Following this process, protein-based tissue substitutes could be reliably fabricated within 60 min (acellular) and within 24 h (cellularized).

Lys-B10, dissolved in molecular grade water at 4° C. at a concentration of 100 mg/mL, was used to embed acellular or cellularized IPN matrices in a sandwich molding setup, FIG. 14. The setup was warmed to 25° C. to allow the liquid elastin mimetic to gel. The IPN elastin composites were then removed from the glass support and trimmed to appropriate dimensions for testing. Long sheets were rolled on 0.9 mm, 1.3 mm and 4 mm ID glass mandrels, kept at 4° C. for 5 min to allow the elastin to go into a liquid state, and warmed to 25° C. to gel the elastin into one contiguous layer.

Generation of Interpenetrating Networks with Tunable Mechanics Dependent on Collagen/Elastin Mixing Ratios and Layering

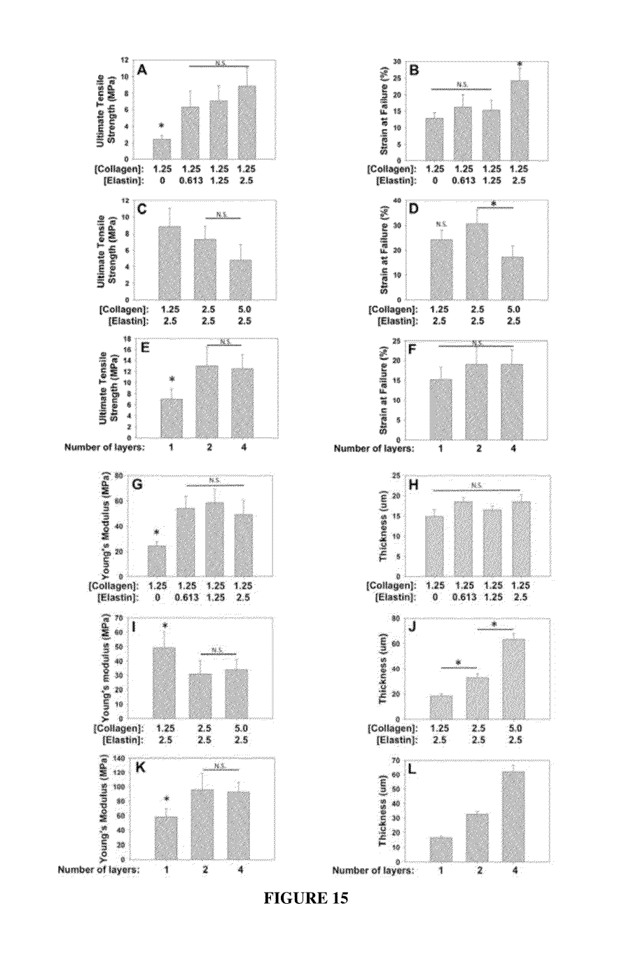

Certain collagen-elastin composites exhibited strengths on the order of 10⁶-10⁷ pascals, comparing superiorly to traditional collagen hydrogels or elastin networks. If initial elastin concentrations were too high, the material resulted in regional inhomogeneities during collagen gelation. Elastin addition to collagen matrices during gelation resulted in a significant increase in strength and stiffness. See FIGS. 15 A and G. Further, an elastin concentration of 2.5 mg/ml and collagen concentration of 1.25 mg/ml and 2.5 mg/ml showed significant increase in strain to failure, over lower collagen-elastin ratios and higher collagen concentrations, FIGS. 15B and D. Increasing collagen concentration from 2.5 mg/ml to 5 mg/ml while maintaining elastin concentration at 2.5 mg/ml in initial gels, resulted in a decrease in UTS and a significant decrease in strain at failure, FIGS. 15 C and D.

Characterization of layered IPNs was performed on 1.25 mg/ml collagen and 1.25 mg/ml elastin matrices. Layered IPNs showed a significant increase in mechanical strength and stiffness from single layer matrices. UTS for single layer matrices (7.03±1.86 MPa) rose significantly for 2 layer and 4 layer constructs (13.0±3.49 MPa and 12.5±2.49 MPa). Similarly Young's modulus rose from 1 layer to 2 and 4 layer constructs (58.4±10.9 MPa, 95.7±23.4 and 92.4±13.5 MPa, respectively). This buttressing effect was limited to strength, and did not significantly decrease strain at failure (10-17%) and stiffness (40-100 MPa), FIG. 15 C, D & I. Thicknesses of matrices had a near linear relationship with initial collagen concentration, showing initial thicknesses of 14.9±1.68 μm for 1.25 mg/ml collagen only and 115±7.45 μm for 4 layered 1.25 mg/ml collagen, 2.5 mg/ml elastin matrices.

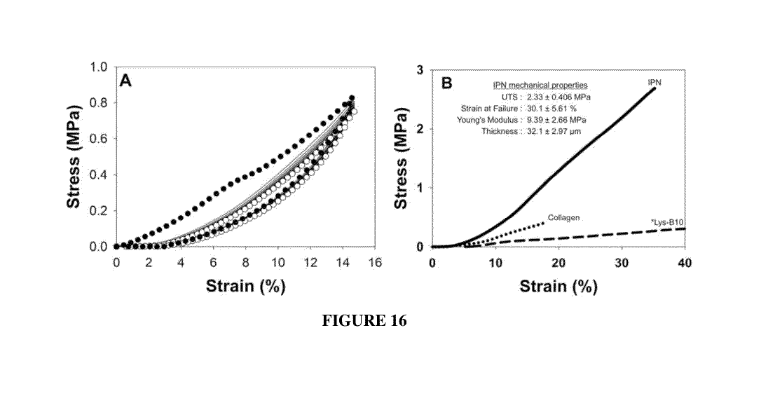

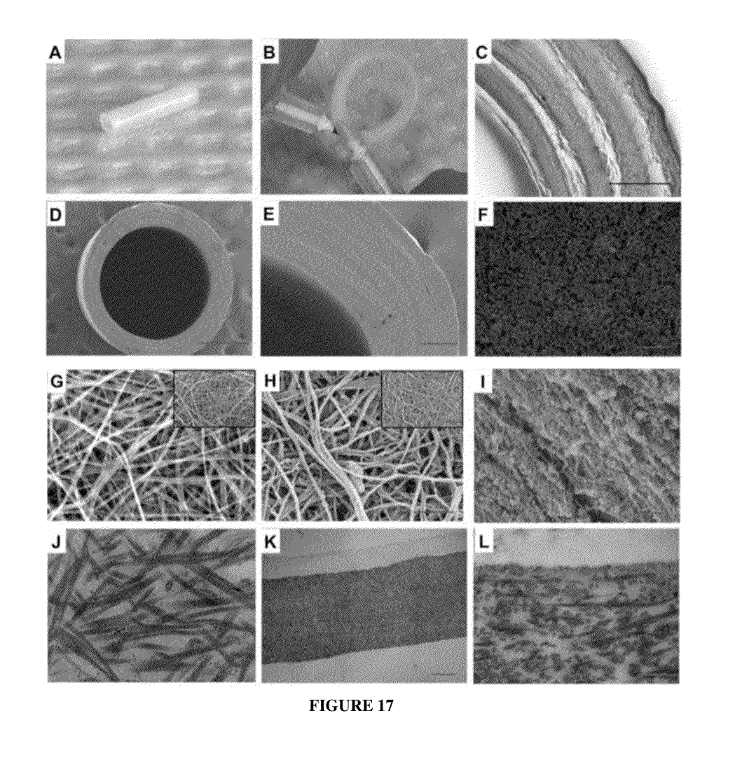

Uncrosslinked IPNs of the aforementioned concentrations were constructed and mechanically tested. The addition of elastin during collagen gelation, increases matrix strength and stiffness, over collagen or elastin alone, and resulted in mechanical properties more closely matching native vascular tissue, FIG. 16 B. UTS of uncrosslinked IPN matrices was 2.33±0.406 MPa, strain to failure was 30.1±5.61% and stiffness was approximately 50% decreased compared to crosslinked matrices, 9.39±2.66 MPa, FIG. 16 B. Resilience, a measure of recovered energy during unloading of matrices, shows much of the energy is recovered during subsequent loading-unloading cycles, comparing favorably to tissue, with minimal energy loss during cyclic loading. The resilience of IPN matrices was 72.9±5.91%, FIG. 16 A. IPN matrices showed enhanced mechanical properties compared to constitutive materials alone. 2.5 mg/ml collagen only matrices had a UTS of 0.474±0.0711 MPa, a strain to failure of 21.1±3.32%, and a Young's Modulus of 2.15±0.690 MPa. LysB10-only constructs showed an UTS of 2.88±0.910 MPa, a strain to failure of 430±34.0% and a Young's Modulus of 0.530±0.0200 MPa.

Biomimetic Vascular Grafts with Mechanical Matching to Native Vasculature

Three mechanical features important for vascular grafts are compliance are burst pressure and suture retention strength. Compliance of 1.3 mm graft and 4 mm grafts closely resembled that of native saphenous vein, 2.36±0.194%/100 mmHg, 2.04±0.330%/100 mmHg, and 0.7-2.6%/100 mmHg, respectively. Burst pressures of tissue engineered grafts were significantly higher than physiologic/pathophysiologic range, 1354±293 mmHg for 1.3 mm grafts and 1237±143 mmHg for 4 mm grafts. Suture retention strength was a function of number of layers within the graft wall. The 1.3 mm grafts had 4-5 layers of composite rolled, FIGS. 16, and 4.0 mm grafts had 8-9 layers. The suture retention strength increased from 38.0±3.46 gF to 72.5±3.59 for 1.3 mm grafts to 4 mm grafts. Further, we have shown the ability to modulate wall thickness as a function of layering/rolling of grafts. 1.3 mm grafts were constructed from a 20 mm composite sheet rolled on a 1.3 mm mandrel. Consequently grafts had a wall thickness of 285±30.4 μm. Similarly, 4 mm grafts were constructed from 100 mm composite sheets rolled on a 4 mm mandrel, and thus had a thicker 602±38.2 μm wall. The observed mechanical strengths approximate or supersede native vasculature and synthetic grafts. See Table 5.

TABLE 5
Mechanical characterization of uncrosslinked 2.5 mg/ml collagen, 2.5 mg/ml
elastin IPN and grafts, compared to native tissue and prosthetic grafts

| | Wall thickness | Compliance | Burst Pressure | Suture retention |
|---|---|---|---|---|
| | (μm) | (%/100 mmHg) | (mmHg) | strength (gF) |
| Implant Graft | 285 ± 30.4 | 2.36 ± 0.194 | 1354 ± 293 | 38.0 ± 3.46 |
| 1.3 mm | | | | |
| Implant Graft | 602 ± 38.2 | 2.04 ± 0.330 | 1237 ± 143 | 72.5 ± 3.59 |
| 4.0 mm | | | | |
| Venous | 250* | 0.7-2.6 | 1600-2500 | 180-250 |
| Arterial | 350-710* | 4.7-17.0 | 2200-4225 | 88-200 |
| Synthetic grafts | 200-600 | 0.2-1.9 | 2580-8270 | 250-1200 |

### Graft Structure and Composition.

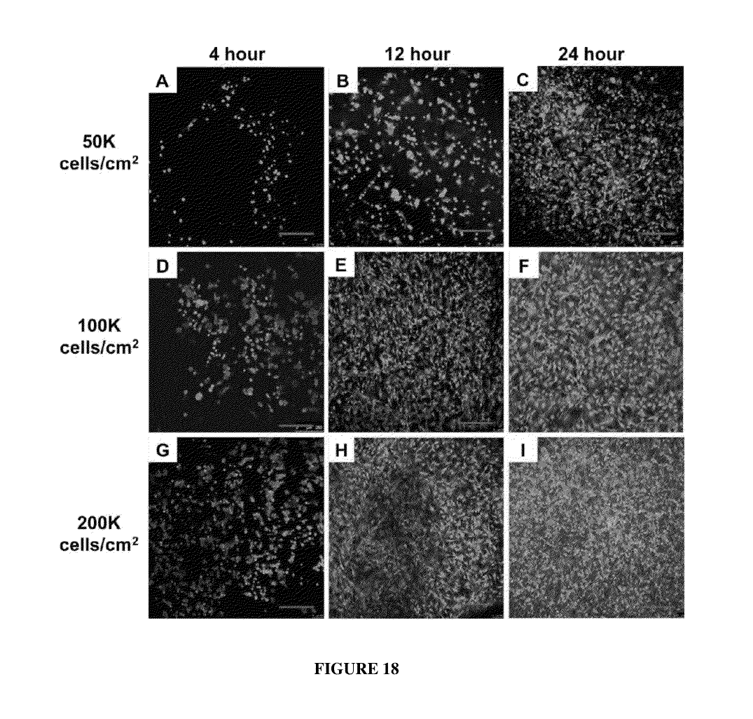

Vascular grafts were generated with a variety of inner diameters using IPNs embedded in an elastin matrix. Dry weight of elastin impregnated sheets shows a significant increase in elastin spatial concentration in constructs, 1620±100 μg/cm², over IPNs alone, 1400±89.8 μg/cm². Compared to collagen matrices alone, which have a spatial concentration of 772±62.0 μg/cm², elastin impregnated IPNs, and resultant grafts are 47% collagen and 53% elastin by dry weight. In vivo studies detailed herein utilize a 1.3 mm ID graft, FIGS. 17 A and B. Van Geison staining of collagen, shows collagen (red) and elastin (yellow) localization in rolled graft, FIG. 17 C. Since red staining is predominant in sections, elastin within IPN structures cannot be visualized optically. Van Geison stained sections show elastin (yellow) coats the lumen of grafts, FIG. 17 C. Ultrastructure of rolled grafts was noted by SEM of critical point dried graft sections. 1.3 mm ID grafts had 4-5 rolled layers and 4 mm ID grafts had 8-9 rolled layers, FIGS. 17 D and E.

The luminal surface has a uniform coating of elastin, including regions where rolling is initiated, FIG. 17 E. Further, the uniform layer of elastin was confirmed by en face visualization of elastin on the luminal surface, which has a distinct fibrillar structure, FIG. 17 F, compared to collagen or IPN matrices, FIGS. 17 G & H.

### Preservation of Fibrillar Collagen Micro- and Ultra-Structure

Native collagen microstructure and ultrastructure maintenance is desirable to avoid premature degradation, immunogenic responses and loss of mechanical integrity. Collagen matrices alone show nanofibrous network formation with collagen fibrils measuring 83.1±9.44 nm, FIG. 17 G. However, when co-gelled with elastin, resulting in IPNs, fibrillar matrices still formed, with collagen fibrils “decorated” with elastin, FIG. 17 H. Collagen fibril diameter increased to 88.1±11.2 nm, but was not significantly different from matrices gelled without the addition of elastin. IPN matrices were then embedded in elastin, in a sandwich molding process that infused elastin into the fibrillar IPN network, filling the nano porous matrix, FIG. 17 I. With the aid of uranyl acetate staining of IPNs, the preservation of D-periodicity within the collagen component is shown in FIG. 17 J. Additionally, after embedding with elastin, fibrillar matrices and D-periodicity is maintained, FIGS. 17 K and L. It is apparent through elastin staining that infusion of collagen mats has occurred with a thin uniform layer of elastin asymmetrically exposed, FIGS. 17 K and L. We have thus shown ability to generate nanofibrous collagen-elastin interpenetrating networks with enhanced mechanical strength, fibrillar networks, and native collagen D-periodicity.

### Mechanical Testing of Planar Composites

To simulate application in planar soft tissues, collagen sheets were cut into 20 mm×5 mm strips and mounted onto a Dynamic Mechanical Thermal Analyzer V (DMTA V, Rheometric Scientific, Piscataway, N.J.) with a gauge length of 10 mm, immersed in PBS at 37° C. Samples were preconditioned 15 times to 66% of the average maximum failure strain of initial test samples, and then tested to failure at 5 mm/min. A total of 8 samples were tested for each group. Thickness of hydrated samples was measured using optical microscopy and then correlated to mechanical data to determine the ultimate tensile strength and strain at failure. Young's modulus was determined by from the slope of the last 4% of the stress strain curve, in addition to ultimate tensile strength (UTS), stain at failure and Young's modulus.

### Mechanical Testing of Tubular Constructs.

Pressure diameter testing to determine compliance and burst pressure of constructs was performed. Tubular collagen-elastin composites were mounted vertically, via luer-lock connectors with a 5 g axial weight, in PBS at 37° C. Grafts were inflated at a rate of 10 mmHg/s, monitored using a pressure transducer (WIKA), and videographed for distention, using a CCD camera. An edge detection program was written in MATLAB to identify and quantify radial distension of grafts based on the outer diameter and correlated to pressure readings. Compliance was determined as the percent difference in outer diameter at systole and diastole, divided by the pressure difference and initial diameter. Grafts were assumed to be incompressible for the range of compliance measurements. The pressure at which the graft started to leak, burst pressure, was also determined, n=4 for 4 mm grafts and n=4 for 1.25 mm grafts. Suture retention strength of grafts was determined by cutting 4 mm×4 mm square sections from planar sheets or longitudinal sections of the graft wall. A 4-0 FS-2 prolene suture (Ethicon) was thrown through the middle of the square segment and pulled in the longitudinal direction using a DMTA (Rheometric Scientific), n=4 for each of 4 grafts. Wall thickness measurements were made on 3 representative cross-sections of each graft. Each graft section was photographed. Image analysis using Adobe Photoshop allowed for the measurement of inner diameter, outer diameter and wall thickness, n=3 for each of 4 grafts.

### Implantation of Grafts in Rat Aortic Interposition Model

Female Sprague-Dawley rats ˜275-300 g (Charles River Labs, Wilmington, Mass.) were anesthetized using isofluorane (2% for induction and 1% for maintenance), shaved, sterilely prepped, and placed on a heating mat at 37° C. A vertical midline abdominal incision was made to expose the infrarenal aorta. Rats received 100 U/kg of heparin prior to aorta clamping through the IVC. The proximal and distal aorta were clamped using microclamps and a segment measuring approximately 1 cm was resected and replaced with an acellular graft using eight to ten interrupted sutures (10-0 Prolene). The abdominal incision was closed with 3-0 Prolene for the fascia and muscular layers, and 4-0 Prolene subcuticular suture for the skin. Rat received clopidogrel 75 mg/kg per day for the first 3 days post-op. Samples (n=8) were explanted at 7 days.

### Histological Analysis to Evaluate Graft Performance

At experimental endpoints, 7 days, rats were anesthetized (2.5% isofluorane induction, 1.5% isofluorane maintenance) and the thoracic cavity was exposed. Whole body fixation was performed. Briefly, an 18 gauge needle was introduced into the left ventricle, and the animal was exsanguinated using 200 mL of saline, and fixed using 200 mL of 10% buffered formalin. Samples were processed for histology. Histology samples were paraffin embedded and sectioned at 5 μm thickness. Evaluation of remodeling of the ECM was determined using Masson's Trichrome.

### Computed Tomography Angiography (CTA) to Evaluate Graft Performance

CTA was performed for 3-dimensional reconstruction and evaluation of patency of the implanted grafts. For each terminal time point (1 week) 4 rats were anesthetized (2.5% isofluorane induction, 1.5% isofluorane maintenance) sterilely prepped and a sternotomy performed to expose the thoracic cavity. To facilitate acquisition of images, whole body exsanguination and fixation were performed and a radiopaque agent (Omnipaque, GE Healthcare, Milwaukee, Wis.) administered. Vessels were then visualized using a NanoSPECT (Bioscan, Washington D.C.) and processed using InVivoScope (Bioscan, Washington D.C.).

### Rapid Cellularization of IPNs Result in Generation of Cellularized Vascular Media Equivalents

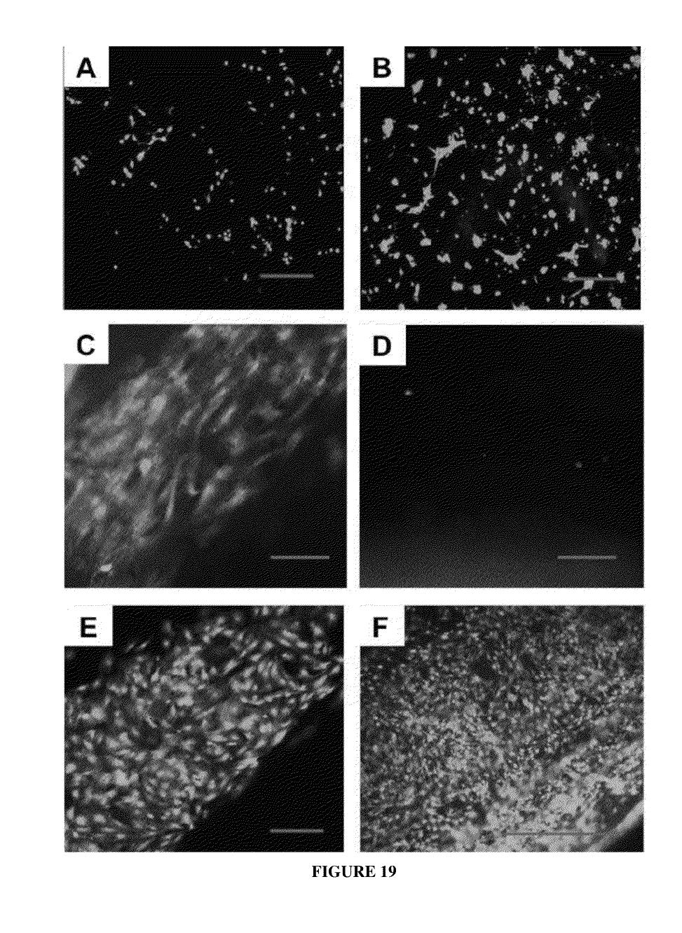

Sterilized IPN matrices were seeded with rMSCs at various concentrations. Live/Dead™ staining showed low cell adhesion at 4 h with absence of filapodia and cell spreading for all concentrations, FIG. 18 A, D and G. Quantification of cellularization at 4 h showed 6.93±2.23%, 16.2±2.57%, 28.1±3.50% confluence for respective spatial seeding densities of 50,000, 100,000 and 200,000 cells/cm². At 12 h, lower cell concentrations show moderate cell adhesion, but high cell concentrations show high cell attachment and spreading, FIG. 18 B, E and H. Quantification of cellularization at 12 h showed 26.3±3.64%, 56.2±4.20%, 84.9±6.28% confluence for respective spatial seeding densities of 50,000, 100,000 and 200,000 cells/cm². At 24 h post seeding, cells seeded at 50,000 cells/cm² witnessed moderate adhesion with cell spreading, but cells seeded at 100,000 and 200,000 cells/cm² showed near confluence, FIG. 18 C, F and I. Quantification of cellularization at 24 h showed 59.6±12.1%, 85.6±6.06%, 87.8±6.35% confluence for respective spatial seeding densities of 50,000, 100,000 and 200,000 cells/cm², respectively. The minimal cell seeding concentration for confluence of IPNs with good cell adhesion and spreading within 24 h was determined (100,000 cells/cm² over 200,000 cells/cm², p=0.82). Consequently, IPNs seeded at 100,000 cells/cm² for 24 h were carried forth to elastin embedding. Cellularized constructs were embedded in elastin using a sandwich molding process, which resulted in a reduction of cells. Imaging of cells immediately after embedding and after 3 days showed cell viability within composites and proliferation, FIGS. 19 A and B. Quantification of cellularization immediately after elastin embedding showed 12.0±3.39% confluence, compared to 3 days post embedding, 25.2±5.47%. In a similar approach, MSCs and ECs we seeded luminally and abluminally (MSCs only) and showed preferential adhesion and near confluence in 24 h of seeding, FIGS. 19 C-F. Cellularized matrices were produced in just over 24 h.

### Small Diameter Vascular Grafts In Vivo

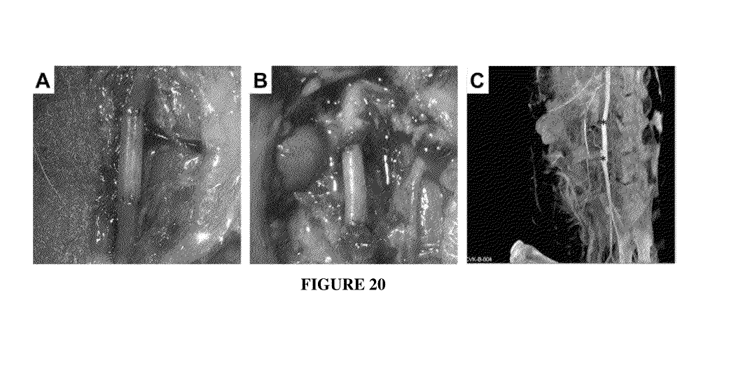

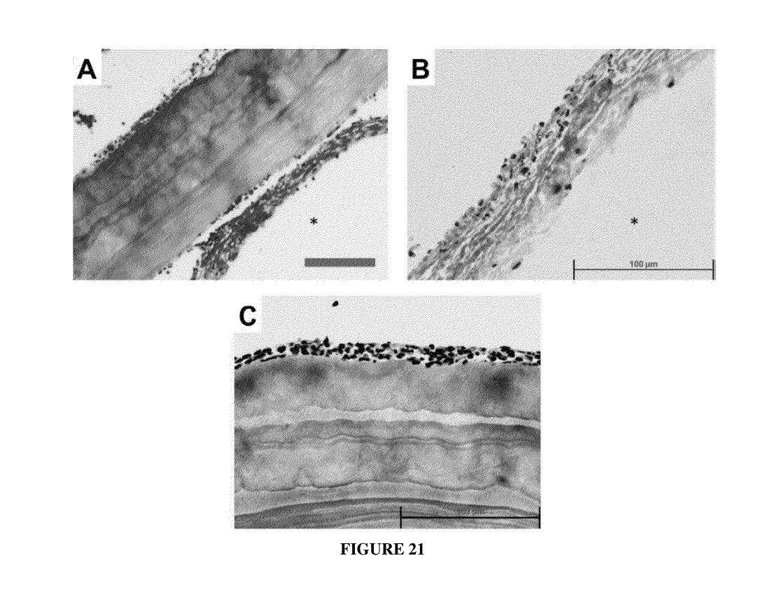

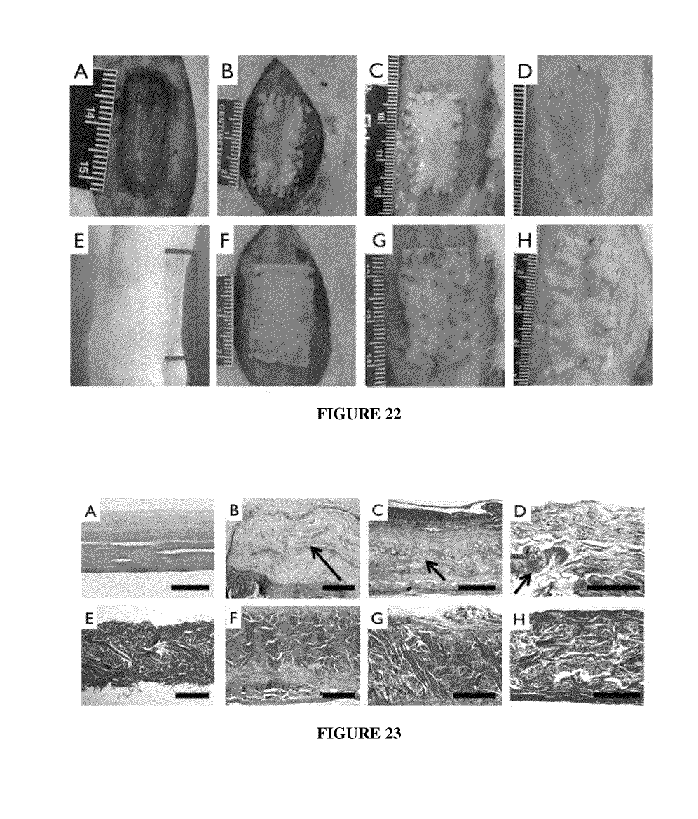

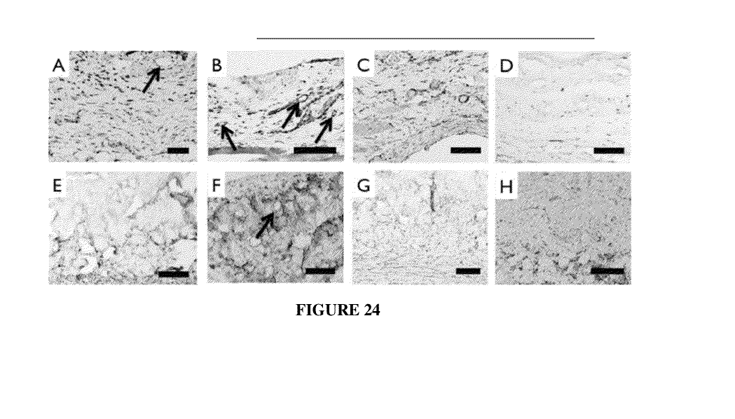

1.3 mm ID vascular grafts were implanted for 1 week in a rat aortic interposition model. Rats were dosed with clopidogrel for 3 days post-op. Grafts could be easily trimmed to desired dimensions for implant (1 cm long), FIG. 20 A. Grafts appeared to be fully perfused upon release of clamps and allowed for visualization of blood flow, FIG. 20 A. Upon explant, grafts appeared patent with minimal adventitial adhesion to abluminal wall, FIG. 20 B. CTA of perfusion fixed interposed grafts show maintenance of graft patency and lack of aneurysmal dilation, FIG. 20 C. Visual observation of graft luminal surface showed no thrombus or visible intimal hyperplasia. Graft integrity was maintained with identifiable staining of ECM based graft components, FIG. 21 A. There appears to be the development of a cellularized neointima which stained positive for collagen, mononuclear cells, and entrapped red blood cells, FIG. 21A, B and C.

### Ventral Hernia Model

A ventral hernia model was created by dissecting the abdominal wall between the xyphoid and pubis to the peritoneum; n=5 per timepoint per group (FIG. 8A). Abdominal wall defects were created in 225-250 g female Wistar rats and repaired with collagen mats or a commercially-available decellularized porcine dermal matrix crosslinked with hexamethylene diisocyanate (HMDI) control implant (1 mm thick Permacol™, Covidien, Mansfield, Mass.). Rats were anesthetized using isoflurane (2.5% induction, 1.5% maintenance); a 5 cm midline incision was made between the xyphoid and pubis. The skin was separated from the muscle layers and a 2.5 cm×1.5 cm incision was made through the muscle layers to the peritoneum.

Multilayer collagen patches or Permacol™ patches, as a reference material, were implanted using an overlay technique. The patch was placed over the defect and sutured in place using 6-0 Prolene™ suture. A 1 cm-long relaxing fascial incision was made 1 cm lateral to either side of the abdominal defect. The skin was closed, and animals were administered pain medication for 48 h.

Animals were sacrificed at 1, 2, and 3 months, the adhesions between the skin and the implant were noted, and changes in implant size measured by photographic analysis. Five (5) rats were used for each group (Permacol™ or collagen) per time point. Harvested samples were removed along with adjacent tissue and fixed in 10% buffered formalin for 24 hours prior to processing. Samples were embedded in paraffin, 5 μm sections obtained and stained for infiltrating cells (Hemotoxylin & Eosin), extracellular matrix production (Masson's Trichrome), monocyte/macrophages (CD68) and endothelial cells, EC (vWF) (Abcam, Cambridge, Mass.). Monocyte/macrophage infiltration was measured by counting positively stained nuclei in 6 random fields for 6 samples at each time point. To measure the strength of integration, 4×20 mm strips of patch and adjacent tissue were excised and mounted on opposing platens of a uniaxial tensile tester (DMTA V, Rheometric Scientific, Piscataway, N.J.) and failure tension determined. Implant area changes were measured from photographs of implants prior to closing and at explantation. Briefly, photographs were taken, as in FIG. 8, the outlines of the implant and explant traced in Image J (NIH, Bethesda, Md.), and compared for each animal.

Neither patch type was associated with re-herniation at time points of up to 3 months (FIG. 8E). The peritoneum was left intact in these studies, so no appreciable adhesion to viscera was noted. Comparison of measurements of implant area with area of explant at each time point showed that both multilayer collagen and control patches showed an increase in area at 3 months (collagen 169±26%, Permacol™ 161±16%, NS). Initial explantation at 1 month showed minimal degradation of either multilayer collagen or control patches (FIG. 8C, G). Conversely, multilayer collagen patches showed a higher level of degradation at 2 and 3 months as compared to Permacol™ (FIG. 8D, H). Tensile strength at the host-patch junction was not significantly different between material types (multilayer collagen: 1 month 1.05±0.24 Nm, 2 month 0.96±0.29 Nm, 3 month 0.98±0.11 Nm; Permacol™: 1 month 1.23±0.32 Nm, 2 month 0.84±0.20 Nm, 3 month 1.03±0.19 Nm).

### Histologic Evaluation

Histologic evaluation was performed using Masson's Trichrome staining (FIG. 9). Prior to implantation, engineered multilayer collagen matrix and Permacol™ matrix showed distinct morphological features. Abdominal muscle stained red and was present adjacent to highly cellularized peritoneal membrane with neo-tissue formation above and below all implants. After the 3-month implant period, ECM staining of collagen showed distinct morphologic differences compared to earlier time points indicating that the multilayer collagen implant had largely been replaced by new collagen deposition (FIG. 9B-D). In contrast, the histomorphology of Permacol™ implants was relatively unchanged over the 3-month implant period (FIG. 9F-H). vWF staining confirmed the development of blood vessels as early as 1 month in both implants (FIG. 10A, B, E, F). CD68 staining revealed a reduction in monocyte/macrophage infiltration during the 3 month time frame, with a greater reduction observed for the multilayer collagen implants as compared to Permacol™ (38.7±8.4% vs. 23.7±5.9%, p<0.05) (FIG. 10C, D, G, H).

1. A synthetic material comprising fibril collagen matrix with a collagen density of greater than 600 micrograms per square centimeter and the collagen fibers maintain D-periodicity.

2. The material of claim 1, wherein the collagen fibers are separated on average by greater than 200 nanometers and less than 1 micrometer.

3. The material of claim 1, wherein the collagen matrix has a greater fibril alignment frequency in one direction.

4. The material of claim 1 is in the form of a sheet with a thickness of less than 50 micrometers.

5. The material of claim 4, wherein the sheet has a continuous surface area of greater than 2 square centimeters.

6. The material of claim 1, further comprising an elastic polymer comprising tetrapeptide, pentapeptide, or hexapeptide repeats comprising proline.

7. The material of claim 6, wherein the elastic polymer comprises peptide repeats of [YaaPUaaXaaZaaₚ]ₙ (SEQ ID NO:1) wherein Yaa is glycine, alanine, lucine, isolucine, or valine; P is Pro; Uaa is glycine, alanine, lucine, isolucine, or valine; Xaa is aspartic acid, glutamic acid, glycine, alanine, lucine, isolucine, or valine or any amino acid except Pro; Zaa is glycine, alanine, lucine, isolucine, or valine; p is 0, 1, 2, 3, 4, 5, or 6; and n is 1 to 1000.

8. The material of claim 7 wherein the elastic polymer comprises a protein copolymer comprising at least one hydrophilic block and at least one hydrophobic block, said copolymer having a first hydrophobic end block, a second hydrophobic end block, and a middle hydrophilic block.

9. The material of claim 8 wherein said middle block comprises [YaaPUaaXaaZaaₚ]ₙ (SEQ ID NO:1) wherein Yaa is glycine, alanine, lucine, isolucine, or valine; P is Pro; Uaa is glycine, alanine, lucine, isolucine, or valine; Xaa is, the same or different at each occurrence; aspartic acid, glutamic acid, glycine, alanine, lucine, isolucine, or valine or any amino acid except Pro; Zaa is glycine, alanine, lucine, isolucine, or valine; p is 0, 1, 2, 3, 4, 5, or 6; and n is 1 to 1000.

10. The material of claim 8 wherein said first and second end blocks comprise [YaaPUaaXaaZaaₚ]ₙ (SEQ ID NO:1) wherein Yaa is glycine, alanine, lucine, isolucine, or valine; P is Pro; Uaa is glycine, alanine, lucine, isolucine, or valine; Xaa is, the same or different at each occurrence; glycine, alanine, lucine, isolucine, or valine; Zaa is glycine, alanine, lucine, isolucine, or valine; p is 0, 1, 2, 3, 4, 5, or 6; and n is 1 to 1000.

11. The material of claim 8, wherein the middle block comprises [(VPGAG)ₚVPGXaaG(VPGAG)_{q}]ₙ (SEQ ID NO:2) wherein Xaa is glutamic acid or aspartic acid, arginine, histidine, lysine, serine, threonine, asparagine, or glutamine; p is 0, 1, 2, or 3; q is 0, 1, 2, or 3; n is 1 to 1000, or n is between 10-100.

12. The material of claim 8, wherein said first and second end blocks comprise [IPAVG]ₙ (SEQ ID NO:3) wherein n is 1 to 200 or 5 to 200.

13. The material of claim 8, wherein the copolymer comprises a peptide sequence comprising lysine between the middle block and the first or second blocks.

14. The material of claim 6, wherein the elastic polymer is within the collagen matrix.

15. A method of making a sheet of collagen comprising
a) mixing an acid solution comprising acid soluble collagen with a buffer under conditions such that a collagen gel forms;
b) incubating the collagen gel in an aqueous buffer solution at a neutral pH for more than one day providing a cured layer of collagen;
c) separating the cured layer of collagen from the buffer solution; and
d) drying the cured layer of collagen to provide dried collagen.

16. The method of claim 15, wherein the collagen gel is stretched to greater than 1, 5, 10 or 20% of the original length in one direction providing a stretched layer of collagen.

17. The method of claim 15, wherein the collagen gel is stretched in the buffer solution.

18. The method of claim 15, wherein the collagen gel is stretched at a speed of less than 200, 100, 50, 10, or 5 micrometers per second.

19. The method of claim 16, wherein the stretched layer of collagen is dried providing a stretched dried layer of collagen.

20. The method of claim 19, further comprising the step of applying a layer of collagen gel to the stretched dried layer of collagen and drying the collagen gel under conditions such that a coated stretched dried layer of collagen forms.

21. The method of claim 19, further comprising the steps of hydrating the stretched dried layer of collagen providing a hydrated stretched layer of collagen, applying a layer of collagen gel to the hydrated stretched layer of collagen and drying the hydrated stretched collagen gel under conditions such that a coated stretched dried layer of collagen forms.

22. The method of claim 19, wherein hydrating the stretched dried layer of collagen is performed on a cylindrical surface wherein the first stretched direction is parallel to the axis of the cylindrical surface.

23. A method of producing a material comprising a layer of collagen and a layer of elastic polymer comprising
a) cooling an acid solution to less than 15 degrees Celsius providing a cooled solution comprising,
acid soluble collagen, and
a protein comprising peptide repeats of [YaaPUaaXaaZaaₚ]ₙ (SEQ ID NO:1)
wherein Yaa is glycine, alanine, lucine, isolucine, or valine;
P is Pro;
Uaa is glycine, alanine, lucine, isolucine, or valine;
Xaa is aspartic acid, glutamic acid, glycine, alanine, lucine, isolucine, or valine or any amino acid except Pro;
Zaa is glycine, alanine, lucine, isolucine, or valine;
p is 0, 1, 2, 3, 4, 5, or 6; and
n is 1 to 1000;
b) neutralizing the cooled solution such that a collagen layer forms; and
c) warming the solution under conditions such that an elastic layer forms.

24. (canceled)

25. A method of producing a material comprising
a) contacting a dried collagen sheet with a solution cooled to less than 15 degree Celsius wherein the cooled solution comprises a protein comprising peptide repeats of [YaaPUaaXaaZaaₚ]ₙ (SEQ ID NO:1) wherein
Yaa is glycine, alanine, lucine, isolucine, or valine;
P is Pro;
Uaa is glycine, alanine, lucine, isolucine, or valine;
Xaa is aspartic acid, glutamic acid, glycine, alanine, lucine, isolucine, or valine or any amino acid except Pro;
Zaa is glycine, alanine, lucine, isolucine, or valine;
p is 0, 1, 2, 3, 4, 5, or 6; and
n is 1 to 1000; and
b) warming the solution under conditions such that an elastic polymer layer forms over the dried collagen sheet.

26. A material made by the process of claim 15.

27. An artificial vascular prosthesis comprising a material as in claim 1.

28. A method of producing a pattern comprising cutting a material as in claim 1.

29-30. (canceled)

31. A material as in claim 1, further comprising a cell.

32. (canceled)

33. A material as in claim 1, further comprising a therapeutic agent.

34. (canceled)

35. A material as in claim 1, further comprising bone granules or minerals, calcium phosphates, hydroxyapatite, tricalcium phosphate, or calcium sulphate.
